(19) United States
(12) Patent Application Publication
(10) Pub. No.: US 2024/0400536 A1
(43) Pub. Date: Dec. 5, 2024

(54) IDEBENONE DERIVATIVES AND THEIR USE IN TREATING PLANTS

(71) Applicant: MITORX THERAPEUTICS LIMITED, Oxford (GB)

(72) Inventors: Matthew WHITEMAN, Exeter (GB); Roberta TORREGROSSA, Exeter (GB); Mark WOOD, Exeter (GB)

(21) Appl. No.: 18/691,650

(22) PCT Filed: Sep. 14, 2022
(86) PCT No.: PCT/GB2022/052325

§ 371 Date: Mar. 13, 2024

(30) Foreign Application Priority Data
Sep. 14, 2021 (GB) 2113117.2

(51) Int. Cl. C07D 339/04; A61K 31/166; A61K 31/167; A61K 31/385; A61P 21/00; C07C 327/48

(52) U.S. Cl. CPC C07D 339/04; A61K 31/166; A61K 31/167; A61K 31/385; A61P 21/00; C07C 327/48

(57) ABSTRACT

The invention relates to a compound of formula (I) comprising a mitochondrial targeting group linked to a group capable of releasing hydrogen sulphide for use in the treatment of the human or animal body or tissues and cells derived therefrom and to the use in the treatment of plants and to novel related compounds.

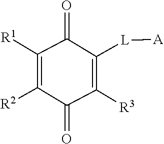

### FIELD

The invention relates to compounds comprising a mitochondrial targeting group linked to a group capable of releasing hydrogen sulphide for use in the treatment of the human or animal body or tissues and cells derived therefrom and to the use in the treatment of plants and to novel related compounds.

### BACKGROUND

### Mitochondria-Targeted H₂S Donor Compounds

In 2014, the first mitochondria-targeted H₂S donor AP39 was reported [Szczesny et al, 2014]. The compound is taken up inside the mitochondria because of its lipophilicity and the positive charge of decyl-TPP⁺. AP39 also showed an increase of intracellular levels of H₂S mainly inside the mitochondria in a concentration-dependent manner, an increase in ATP production in endothelial cells, as well as an increase of protein persulfidation inside the mitochondria. However, AP39 is hygroscopic, has poor aqueous solubility, potential toxicity issues and has not been developed as a drug.

### Idebenone

Coenzyme Q10 (CoQ10) or ubiquinone, exerts redox and antioxidant effects due to the presence of 1,4-benzoquinone ring. CoQ10 also has the ability to interact with other redox carriers in the mitochondrial electron transport chain [Escribano-Lopez et al, 2019]. To obtain analogues, with the same antioxidant properties but with a better bioavailability, idebenone was developed by Takeda Chemical Industries (Osaka Japan) and launched in the market as a medicine against age-related brain dysfunction, in 1986 [Sugiyama and Fujita, 1985]. No research has been performed to use the mitochondrial targeting properties of idebenone and derivatives to target H₂S donors to the mitochondria.

A number of idebenone derivatives have been made as antioxidants:

| Structure | IUPAC | Patent/app |
|---|---|---|
| 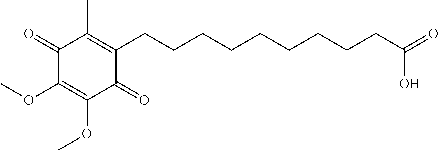 [SMILES: COC1=C(OC)C(=O)C(CCCCCCCCCC(=O)O)=C(C)C1=O] | 10-(4,5-dimethoxy-2- methyl-3,6- dioxocyclohexa-1,4-dien-1- yl)decanoic acid | U.S. Pat. No. 4,139,545 |
| 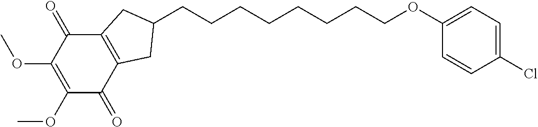 [SMILES: COC1=C(OC)C(=O)C2=C(CC(CCCCCCCCOc3ccc(Cl)cc3)C2)C1=O] | 2-[8-(4-chlorophenoxy) octyl]-5,6- dimethoxy-2,3-dihydro- 1H-indene-4,7- dione | U.S. Pat. No. 6,326,369 |
| 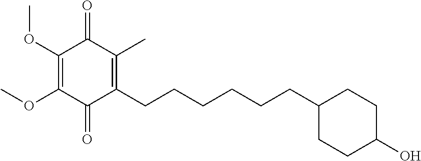 [SMILES: COC1=C(OC)C(=O)C(CCCCCCC2CCC(O)CC2)=C(C)C1=O] | 2-[6-(4-hydroxycyclohexyl) hexyl]-5,6- dimethoxy-3- methylcyclohexa-2,5- diene-1,4-dione | U.S. Pat. No. 4,897,420 |
| 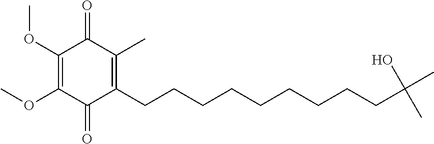 [SMILES: COC1=C(OC)C(=O)C(CCCCCCCCCC(C)(C)O)=C(C)C1=O] | 2-(10-hydroxy-10- methylundecyl)-5,6- dimethoxy-3- methylcyclohexa-2,5- diene-1,4-dione | EP0201196 |

A number of idebenone derivatives have also been made as donors of the gasotransmitter nitric oxide:

| Structure | IUPAC | Patent/app |
|---|---|---|
| 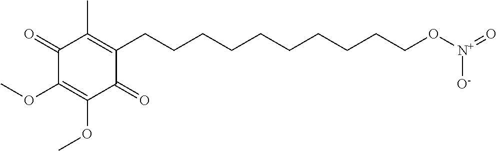 [SMILES: COC1=C(OC)C(=O)C(CCCCCCCCCCO[N+](=O)[O-])=C(C)C1=O] | 10-(4,5-dimethoxy- 2-methyl-3,6- dioxocyclohexa- 1,4-dien-1-yl)decyl nitrate | U.S. Pat. No. 9,446,015 |
| 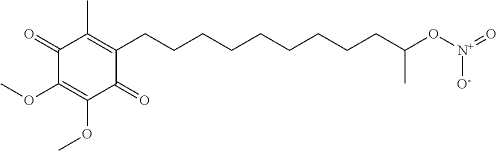 [SMILES: COC1=C(OC)C(=O)C(CCCCCCCCCC(C)O[N+](=O)[O-])=C(C)C1=O] | 1-(4,5-dimethoxy- 2-methyl-3,6- dioxocyclohexa-1,4- dien-1-yl)undecan- 2-yl nitrate | U.S. Pat. No. 9,061,962 |
| 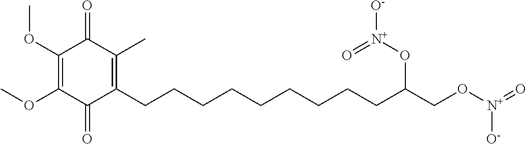 [SMILES: COC1=C(OC)C(=O)C(CCCCCCCCCC(CO[N+](=O)[O-])O[N+](=O)[O-])=C(C)C1=O] | [11-(4,5-dimethoxy- 2-methyl-3,6- dioxocyclohexa- 1,4-dien-1-yl)-1- nitrooxyundecan- 2-yl]nitrate | U.S. Pat. No. 9,061,962 |
| 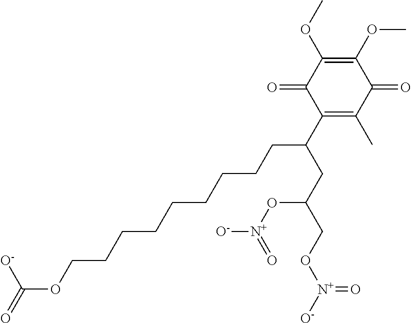 [SMILES: COC1=C(OC)C(=O)C(C(CCCCCCCCCOC(=O)[O-])CC(CO[N+](=O)[O-])O[N+](=O)[O-])=C(C)C1=O] | [10-(4,5-dimethoxy- 2-methyl-3,6- dioxocyclohexa- 1,4-dien-1-yl)-12,13- dinitrooxytridecyl] carbonate | WO2015155234 |
| 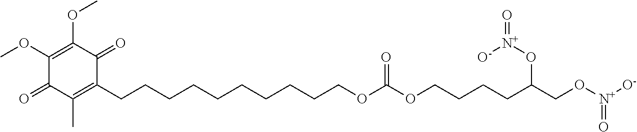 [SMILES: COC1=C(OC)C(=O)C(CCCCCCCCCCOC(=O)OCCCCC(CO[N+](=O)[O-])O[N+](=O)[O-])=C(C)C1=O] | 2-{10-[({[5,6- bis(nitrooxy)hexyl] oxy}carbonyl)oxy] decyl}-5,6-dimethoxy- 3-methylcyclohexa- 2,5-diene-1,4-dione | WO2015155234 |
| 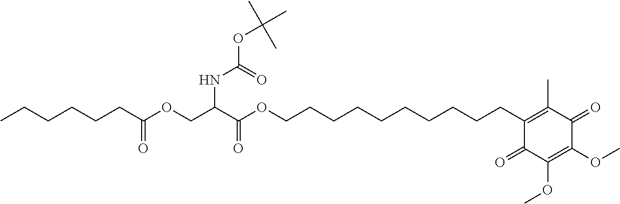 [SMILES: CCCCCCC(=O)OCC(NC(=O)OC(C)(C)C)C(=O)OCCCCCCCCCCC1=C(C)C(=O)C(OC)=C(OC)C1=O] | 2-{10-[({[5,6-bis (nitrooxy)hexyl]oxy} carbonyl)oxy)decyl}- 5,6-dimethoxy-3- methylcyclohexa- 2,5-diene-1,4-dione | WO2015155234 |
| 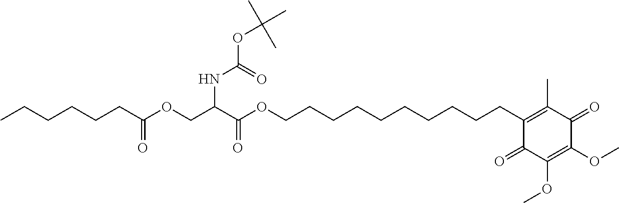 [SMILES: CCCCCCC(=O)OCC(NC(=O)OC(C)(C)C)C(=O)OCCCCCCCCCCC1=C(C)C(=O)C(OC)=C(OC)C1=O] | [3-[10-(4,5- dimethoxy-2- methyl-3,6- dioxocyclohexa-1,4- dien-1-yl)decoxy]- 2-[(2-methylpropan-2- yl)oxycarbonylamino]- 3-oxopropyl] heptanoate | WO2015155234 |
| 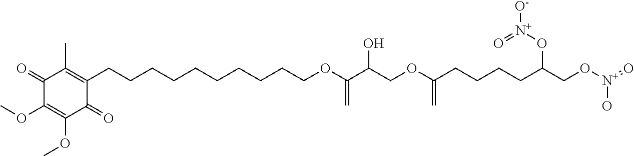 | [2-amino-3-[10- (4,5-dimethoxy-2- methyl-3,6-dioxo- cyclohexa-1,4-dien-1- yl)decoxy]-3- oxopropyl] 6,7- dinitrooxyheptanoate | WO2015155234 |
| 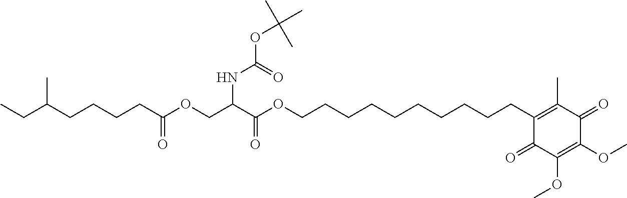 [SMILES: CCC(C)CCCCC(=O)OCC(NC(=O)OC(C)(C)C)C(=O)OCCCCCCCCCCC1=C(C)C(=O)C(OC)=C(OC)C1=O] | [3-[10-(4,5- dimethoxy-2- methyl-3,6- dioxocyclohexa- 1,4-dien-1-yl)decoxy]- 2-[(2-methylpropan-2- yl)oxycarbonylamino]- 3-oxopropyl] 6- methyloctanoate | WO2015155234 |

There remains a pressing unmet clinical need for H₂S donating molecules with improved properties which are targeted towards the mitochondria.

“In Vitro Antioxidant Activity of Idebenone Derivative-Loaded Solid Lipid Nanoparticles” Lucia Montenegro et al., Molecules 2017, 22, 887 discloses idebenone derivatives for the treatment of neurodegenerative diseases involving mitochondria dysfunctions.

“Coenzyme Q Functionalized CdTe/ZnS Quantum Dots for Reactive Oxygen Species (ROS) Imaging”, Li-Xia Qin et al., Chem. Eur. J. 2011, 17, 5262-5271 discloses CoQ derivatived QDs as probes to image redox coenzyme function in vitro and in vivo.

### SUMMARY OF THE INVENTION

The present invention provides active compounds, specifically, mitochondrially targeted H₂S donors, as described herein.

The term “active,” as used herein, specifically includes both compounds with intrinsic activity (drugs) as well as prodrugs of such compounds, which prodrugs may themselves exhibit little or no intrinsic activity. One aspect of the invention pertains to active H₂S donating compounds, as described herein, which are targeted towards the mitochondrion.

According to a first aspect of the present invention, there is provided a compound of formula (I):

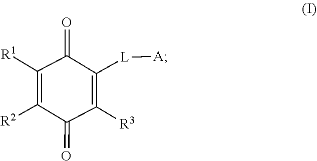

wherein R¹ and R² are independently selected from a C₁₋₆ alkyl group, a C₁₋₆ alkoxy group or together form a cycloalkyl or aryl ring:
wherein R³ is an C₁₋₆ alkyl or C₁₋₆ alkoxy group;
wherein L is a linker group;
wherein A is a group capable of releasing hydrogen sulphide;
or a pharmaceutically acceptable salt thereof.

The inventors have found that the compounds of formula (I) may provide effective treatments for neuromuscular or muscular conditions, particularly those mediated by mitochondrial H₂S donors (mtH2SD) by targeting mitochondria through the 1,4-benzoquinone ring and releasing hydrogen sulphide in the mitochondria to produce the desired physiological effects.

According to a second aspect of the present invention, there is provided a compound according to the first aspect for use as a medicament.

According to a third aspect of the present invention, there is provided a compound according to the first aspect for use in the treatment of a neuromuscular or muscular condition.

The neuromuscular or muscular condition may be mediated by mtH2SD. The neuromuscular or muscular condition may be selected from Duchenne Muscular dystrophy, COPD, Leigh syndrome, primary mitochondrial disease, Pancreatic islet transplant, Pre-eclampsia, Cardiac transplant, Renal transplant, Cardiovascular dysfunction, Blunt chest trauma and haemorrhagic shock, Necrotizing enterocolitis, Myocardial reperfusion injury, Burn injury, Diabetic vascular disease, Alzheimer's disease, Acute renal injury, Neurological damage post cardiac arrest and Hypertension.

Suitably the compound according to the first aspect is for use in the treatment of a disease involving mitochondrial dysfunction, such as the diseases/conditions listed above.

According to a fourth aspect of the present invention, there is provided a pharmaceutical composition comprising a compound according to the first aspect, or a pharmaceutically acceptable salt thereof, and a pharmaceutically acceptable carrier, excipient, or diluent. The pharmaceutical composition may be for use in the treatment of a neuromuscular or muscular condition.

According to a fifth aspect of the present invention, there is provided a method of prevention, management and/or treatment of a neuromuscular or muscular condition in a subject in need thereof, the method comprising administering to the subject a therapeutically effective amount of a compound according to the first aspect or a pharmaceutical composition according to fourth aspect.

Another aspect of the invention pertains to active compounds, as described herein, which treat a neuromuscular or muscular condition, such as muscular dystrophy.

Another aspect of the invention pertains to active compounds, as described herein, which treat conditions which are known to be mediated by mtH2SD, or which are thought to be treatable by a mtH2SD (such as, e.g., AP39).

Another aspect of the present invention pertains to a composition comprising a compound as described herein and a pharmaceutically acceptable carrier.

Another aspect of the present invention pertains to methods of H₂S donation in a cell, comprising contacting said cell with an effective amount of an active compound, as described herein.

Another aspect of the present invention pertains to methods of donating H₂S, comprising contacting a cell with an effective amount of an active compound, as described herein, whether in vitro or in vivo.

Another aspect of the present invention pertains to methods of treating a condition in a patient comprising administering to said patient a therapeutically-effective amount of an active compound, as described herein. In one suitable embodiment, the condition is muscular dystrophy. In one suitable embodiment, the condition is Duchenne Muscular Dystrophy.

Another aspect of the present invention pertains to methods of treating a condition in a patient which is known to be mediated by mtH2SD, or which is thought to be treatable by mH2SD (such as, e.g., AP39, comprising administering to said patient a therapeutically-effective amount of an active compound, as described herein.

Another aspect of the present invention pertains to an active compound, as described herein, for use in a method of treatment of the human or animal body.

Another aspect of the present invention pertains to use of an active compound, as described herein, for the manufacture of a medicament for use in the treatment of a neuromuscular or muscular condition. In one suitable embodiment, the proliferative condition is muscular dystrophy.

In one suitable embodiment, the proliferative condition is Duchenne Muscular Dystrophy.

In one suitable embodiment, the proliferative condition is COPD (chronic obstructive pulmonary disease).

Another aspect of the present invention pertains to use of an active compound for the manufacture of a medicament, for example, for the treatment of conditions which are known to be mediated by mtH2SD, or which are known to be treated by mtH2SD (such as, e.g., AP39), as discussed herein. Such conditions include but are not limited to the following:
Duchenne Muscular dystrophy (Ellwood et al 2021)
COPD, airway inflammation (Karaman et al, 2020)
Leigh syndrome, primary mitochondrial disease (Fox et al, 2020)
Pancreatic islet transplant (Nishime et al 2020)
Pre-eclampsia (Sanchez-Aranguren et al, 2020; Covarrubias et al 2019)
Cardiac transplant (Zhu et al 2019)
Renal transplant (Juriasingani et al, 2018; Lobb et al 2017)
Cardiovascular dysfunction (Latorre et al, 2018)
Blunt chest trauma and haemorrhagic shock (Wepler et al, 2019)
Necrotizing enterocolitis (Drucker et al, 2018)
Myocardial reperfusion injury (Karwi et al 2017; Chatzianastasiou et al, 2016)
Burn injury (Ahmad et al, 2016)
Diabetic vascular disease (Gero et al 2016)
Alzheimer's disease (Zhao et al 2016)
Acute renal injury (Ahmad et al 2016)
Neurological damage post cardiac arrest (Ikeda et al, 2015)
Hypertension (Tomasova 2015)

Another aspect of the present invention pertains to a kit comprising (a) the active compound, preferably provided as a pharmaceutical composition and in a suitable container and/or with suitable packaging; and (b) instructions for use, for example, written instructions on how to administer the active compound.

Another aspect of the present invention pertains to compounds obtainable by a method of synthesis as described herein, or a method comprising a method of synthesis as described herein.

Another aspect of the present invention pertains to compounds obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

The present invention also provides methods of producing H₂S in the mitochondria of a cell, comprising contacting said cell with an effective amount of an active compound. Such a method may be practised in vitro or in vivo. In one embodiment, the method is performed in vitro. In one embodiment, the method is performed in vivo. Preferably, the active compound is provided in the form of a pharmaceutically acceptable composition.

One of ordinary skill in the art is readily able to determine whether or not a candidate compound counteracts mitochondrial dysfunction. For example, one assay which may conveniently be used in order to assess the level of mitochondrial dysfunction offered by a particular compound is described in the examples below.

For example, a sample of cells may be grown in vitro and an active compound brought into contact with said cells, and the effect of the compound on those cells observed. As an example of “effect,” the morphological status of the cells (e.g., alive or dead, etc.) may be determined. Where the active compound is found to exert an influence on the cells, this may be used as a prognostic or diagnostic marker of the efficacy of the compound in methods of treating a patient carrying cells of the same cellular type.

### Methods of Treatment

The invention further provides methods of treatment, comprising administering to a subject in need of treatment a therapeutically-effective amount of an active compound, preferably in the form of a pharmaceutical composition.

The invention further provides active compounds for use in a method of treatment of the human or animal body by therapy, for example, in the treatment of a condition mediated by H₂S donors, a condition known to be treated by H₂S donors (e.g. AP39), or other condition as described herein.

The invention further provides the use of an active compound for the manufacture of a medicament, for example, for the treatment of a condition mediated by mitochondrial dysfunction, or a condition known to be treated by compounds known to counteract mitochondrial dysfunction (such as, e.g., AP39).

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio.

The term “treatment” includes combination treatments and therapies, in which two or more treatments or therapies are combined, for example, sequentially or simultaneously. Examples of treatments and therapies include, but are not limited to small molecules, gene therapy, cell therapy, antibody therapy.

Active compounds may also be used, as described above, in combination therapies, that is, in conjunction with other agents, for example, steroids.

The present invention also provides active compounds which counteract mitochondrial dysfunction and which treat a condition mediated by mitochondrial dysfunction.

The term “a condition mediated by mitochondrial dysfunction,” as used herein pertains to a condition in which mitochondrial dysfunction is important or necessary, e.g., for the onset, progress, expression, etc. of that condition, or a condition which is known to be treated by compounds which counteract mitochondrial dysfunction such as e.g. AP39.

One of ordinary skill in the art is readily able to determine whether or not a candidate compound treats a condition involving mitochondrial dysfunction for any particular cell type. For example, assays which may conveniently be used to assess the activity offered by a particular compound are described in the examples below.

The present invention also provides active compounds which are mitochondrial H₂S donors and treat diseases involving mitochondrial dysfunction. A non-limiting list of such indications is given above.

### Routes of Administration

The active compound or pharmaceutical composition comprising the active compound may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrastemal; by implant of a depot or reservoir, for example, subcutaneously or intramuscularly.

### The Subject

The subject may be a prokaryote (e.g., bacteria) or a eukaryote (e.g., protoctista, fungi, plants, animals).

The subject may be an animal, a mammal, a placental mammal, a marsupial, a monotreme a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey an ape or a human.

Furthermore, the subject may be any of its forms of development, for example, a spore, a seed, an egg, a larva, a pupa, or a foetus.

Suitably, the subject is a human.

### Formulations

While it is possible for the active compound to be used (e.g., administered) alone, it is often preferable to present it as a formulation.

Thus, one aspect of the present invention pertains to a composition comprising a compound, as described herein, and a carrier.

In one embodiment, the composition is a pharmaceutical composition (e.g., formulation, preparation, medicament) comprising a compound, as described herein, and a pharmaceutically acceptable carrier.

In one embodiment, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 18th edition, Mack

Publishing Company, Easton, Pa., 1990; and Handbook of Pharmaceutical Excipients, 2nd edition, 1994. Another aspect of the present invention pertains to methods of making a pharmaceutical composition comprising admixing at least one active compound, as defined above, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio.

Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, lozenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more active compounds and optionally one or more other pharmaceutically acceptable ingredients, including, for example, penetration, permeation, and absorption enhancers. Formulations may also suitably be provided in the form of a depot or reservoir.

The active compound may be dissolved in, suspended in, or admixed with one or more other pharmaceutically acceptable ingredients. The active compound may be presented in a liposome or other microparticulate which is designed to target the active compound, for example, to blood components or one or more organs.

Formulations suitable for oral administration (e.g, by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, lozenges, pastilles, as well as patches, adhesive plasters, depots, and reservoirs. Lozenges typically comprise the active compound in a flavored basis, usually sucrose and acacia or tragacanth. Pastilles typically comprise the active compound in an inert matrix, such as gelatin and glycerin, or sucrose and acacia. Mouthwashes typically comprise the active compound in a suitable liquid carrier.

Formulations suitable for sublingual administration include tablets, lozenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, lozenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the active compound and a paraffinic or a water-miscible ointment base.

Creams are typically prepared from the active compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions are typically prepared from the active compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono-or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the active compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the active compound is dissolved or suspended in a suitable carrier, especially an aqueous solvent for the active compound.

Formulations suitable for rectal administration may be presented as a suppository with a suitable base comprising, for example, natural or hardened oils, waxes, fats, semi-liquid or liquid polyols, for example. cocoa butter or a salicylate; or as a solution or suspension for treatment by enema.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the active compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection.

Typically, the concentration of the active compound in the liquid is from about 1 ng/ml to about 10 μg/ml, for example from about 10 ng/ml to about 1 μg/ml. The formulations may be presented in unit-dose or multi- dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the active compounds, and compositions comprising the active compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

### Kits

One aspect of the invention pertains to a kit comprising (a) the active ingredient, preferably provided in a suitable container and/or with suitable packaging; and (b) instructions for use, for example, written instructions on how to administer the active compound, etc.

The written instructions may also include a list of indications for which the active ingredient is a suitable treatment.

As will be appreciated by one of skill in the art, features and suitable embodiments of one aspect of the invention will also pertain to other aspects of the invention.

The active compounds

The compounds of the first aspect have the formula (I):

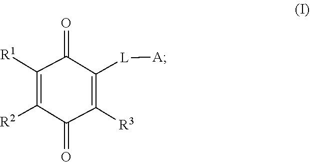

wherein R¹ and R² are independently selected from a C₁₋₆ alkyl group, a C1e alkoxy group or together form a cycloalkyl or aryl ring;
wherein R³ is an C₁₋₆ alkyl or C₁₋₆ alkoxy group;
wherein L is a linker group;
wherein A is a group capable of releasing hydrogen sulphide;
or a pharmaceutically acceptable salt thereof.

In some embodiments R¹ and R² are both C₁₋₆ alkoxy groups, suitably C₁₋₃ alkoxy groups, suitably-OMe. Suitably R³ is an C₁₋₆ alkyl group, suitably a C₁₋₃ alkoxy group.

Suitably R¹ and R² are both-OMe and R³ is an C₁₋₃ alkyl group.

When R¹ and R² together form a cycloalkyl or aryl ring, the cycloalkyl or aryl ring is suitably a 5-, 6-and 7-membered cycloalkyl or aryl ring, suitably a 5-, 6-and 7-membered aryl ring. The cycloalkyl or aryl ring may be optionally substituted, suitably with one or more of C₁-C₄ alkyl, C₁-C₄ alkoxy, C₁-C₄ alkylamino, C₁-C₄ alkylthio, hydroxy, amino, nitro, thiol, chloro, fluoro, bromo, CF₃, CHF₂ or CH₂F groups.

In some embodiments R¹ and R² form a 5-or 6-membered aryl ring, suitably a 6-membered aryl ring. Therefore in such embodiments, the compound comprises a 1,4-naphthoquinone group.

Suitably the group capable of releasing hydrogen sulphide A is selected from:

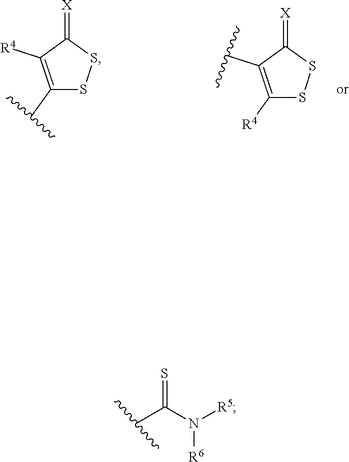

wherein X is S, O or N—OH and R⁴, R⁵ and R⁶ are independently selected from H or C₁₋₇ alkyl groups. The C₁₋₇ alkyl groups may be optionally substituted, suitably with one or more of C₁-C₄ alkyl, C₁-C₄ alkoxy, C₁-C₄ alkylamino, C₁-C₄ alkylthio, hydroxy, amino, nitro, thiol, chloro, fluoro, bromo, CF₃, CHF₂ or CH₂F groups. Suitably X is S or O.

Suitably R⁴ is H.

Suitably R⁵ and R⁶ are methyl or H, suitably H.

Suitably group A is selected from a thiocarbamoyl group, a 5-thioxo-5H-1,2-dithiol-3-yl group, a 5-thioxo-5H-1,2-dithiol-4-yl group, a 5-oxo-5H-1,2-dithiol-3-yl group, a 5-oxo-5H-1,2-dithiol-4-yl group, a 5-hydroxyimino-5H-1,2-dithiol-3-yl group, a 5-hydroxyimino-5H-1,2-dithiol-4-yl group, a phosphinodithioate group or a phosphinodithioic acid group.

Suitably A is selected from the following groups:

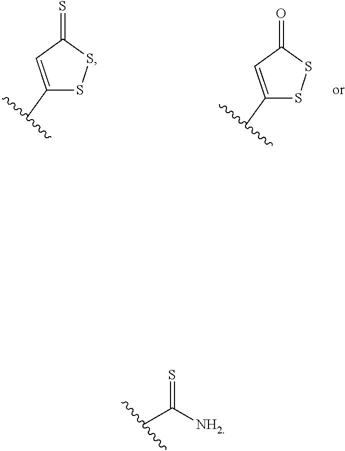

Suitably the linker group L comprises a group B which is an optionally substituted alkyl chain, optionally substituted alkenyl chain, or optionally substituted alkynyl chain. Suitably B is an unsubstituted C₁₋₂₀ alkyl chain, suitably a C₆₋₁₄ alkyl chain, suitably a C₈₋₁₂ alkyl chain.

Suitably the linker group L comprises a group Z, wherein Z is selected from a direct bond, —C(═O)NH—, —NHC(═O)—, —O—, —S—, —S(═O)₂NH—, —NHS(═O)₂—, —OC(═O)—, —OC(═O)CH₂O— and —C(═O)O—. Suitably Z is —C(═O)O— or —OC(═O)CH₂O—, suitably —C(═O)O—. In addition, Z may be the group —OCH₂C(═O)O—.

Suitably the linker group L comprises a group Y which is an optionally substituted 5 or 6 membered cycloalkyl or aryl ring. Suitably Y is an optionally substituted phenyl group and wherein groups Z and A are attached para to each other on the phenyl group (i.e. in a 1,4 arrangement). Suitably Y is an unsubstituted phenyl group and groups Z and A are attached para to each other on the phenyl group.

The Y group may be optionally substituted with one or more of C₁—C₄ alkyl, C₁—C₄ alkoxy, C₁—C₄ alkylamino, C₁-C₄ alkylthio, hydroxy, amino, nitro, thiol, chloro, fluoro, bromo, CF₃, CHF₂ or CH₂F groups.

Suitably the compound according to the first aspect has the formula (II):

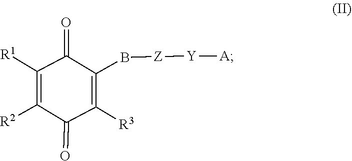

wherein R¹, R² and R³ are as defined above;
wherein B is an optionally substituted alkyl chain, optionally substituted alkenyl chain, or optionally substituted alkynyl chain;
wherein Z is selected from wherein Z is selected from a direct bond, —C(═O)NH—, —NHC(═O)—, —O—, —S—, —S(═O)₂NH—, —NHS(═O)₂—, —OC(═O)—, —OC(═O)CH₂O— and —C(═O)O—;
wherein Y is an optionally substituted 5 or 6 membered cycloalkyl or aryl ring;
wherein A is a group capable of releasing hydrogen sulphide;
or a pharmaceutically acceptable salt thereof.

Suitably B, Z, Y and A are as defined above.

Where groups are noted as being “optionally substituted”, said groups are suitably optionally substituted with one or more groups as defined below and referred to as “R” groups. Suitably said groups are optionally substituted with one or more halogens, one or more aryl groups, one or more C₁₋₆ alkyl groups or one or more C₁₋₆ alkoxy groups.

In some embodiments, the compound according to the first aspect has the formula (II):

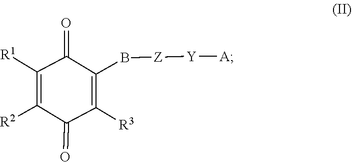

wherein R¹ and R² are both C₁₋₆ alkoxy groups or together form a 6-membered aryl ring;
wherein R³ is an C₁₋₆ alkyl group.
wherein B is an optionally substituted a C₆₋₁₄ alkyl chain;
wherein Z is a group selected from −C(═O)NH—, —NHC(═O)—, —O—, —OC(═O)—, —OC(═O)CH₂O—, —OCH₂C(═O)O—and —C(═O)O—;
wherein Y is an optionally substituted phenyl group wherein groups Z and A are attached para to each other on the phenyl group;
wherein A is selected from:

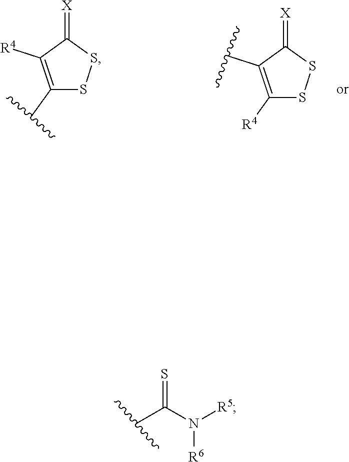

wherein X is S, O or N—OH and R⁴, R⁵ and R⁶ are independently selected from H or C₁₋₇ alkyl groups; or a pharmaceutically acceptable salt thereof.

In some embodiments, the compound according to the first aspect has the formula (II):

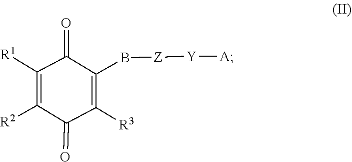

wherein R¹ and R² are both C₁₋₈ alkoxy groups or together form a 6-membered aryl ring;
wherein R³ is an C₁₋₆ alkyl group.
wherein B is a C₆₋₁₄ alkyl chain optionally substituted with one or more of C₁-C₄ alkyl, C_{1—}C₄ alkoxy, C_{1—}C₄ alkylamino, C_{1—}C₄ alkylthio, hydroxy, amino, nitro, thiol, chloro, fluoro, bromo, CF₃, CHF₂ or CH₂F groups.;
wherein Z is a group selected from −C(═O)NH—, —NHC(═O)—, —O—, —OC(═O)—, —OC(═O)CH₂O—, —OCH₂C(═O)O— and—C(═O)O—;
wherein Y is a phenyl group wherein groups Z and A are attached para to each other on the phenyl group, optionally substituted with one or more of C₁-C₄ alkyl, C₁-C₄ alkoxy, C₁-C₄ alkylamino, C₁-C₄ alkylthio, hydroxy, amino, nitro, thiol, chloro, fluoro, bromo, CF₃, CHF₂ or CH₂F groups.;
wherein A is selected from:

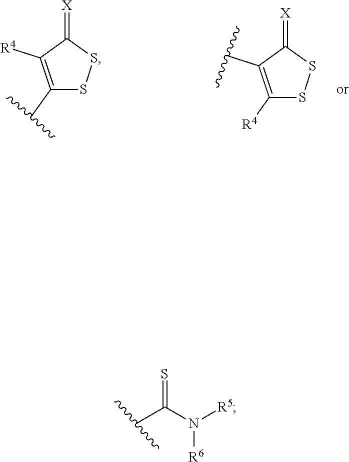

wherein X is S, O or N—OH and R⁴, R⁵ and R⁶ are independently selected from H or C₁₋₇ alkyl groups; or a pharmaceutically acceptable salt thereof.

In some embodiments, the compound according to the first aspect has the formula (II):

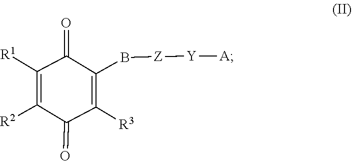

wherein R¹ and R² are both C₁₋₆ alkoxy groups or together form a 6-membered aryl ring;
wherein R³ is an C₁₋₈ alkyl group.
wherein B is an optionally substituted a C₈₋₁₄ alkyl chain;
wherein Z is selected from a —OC(═O)—, —OC(═O)CH₂O— and—C(═0)O—;
wherein Y is an optionally substituted phenyl group wherein groups Z and A are attached para to each other on the phenyl group;
wherein A is selected from:

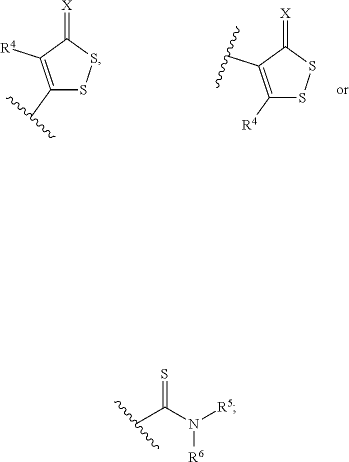

wherein X is S, O or N—OH and R⁴, R⁵ and R⁸ are independently selected from H or C₁₋₇ alkyl groups;

or a pharmaceutically acceptable salt thereof.

In some embodiments, the compound according to the first aspect is selected from Examples 1-17 described below.

In some embodiments, the compound according to the first aspect is selected from:

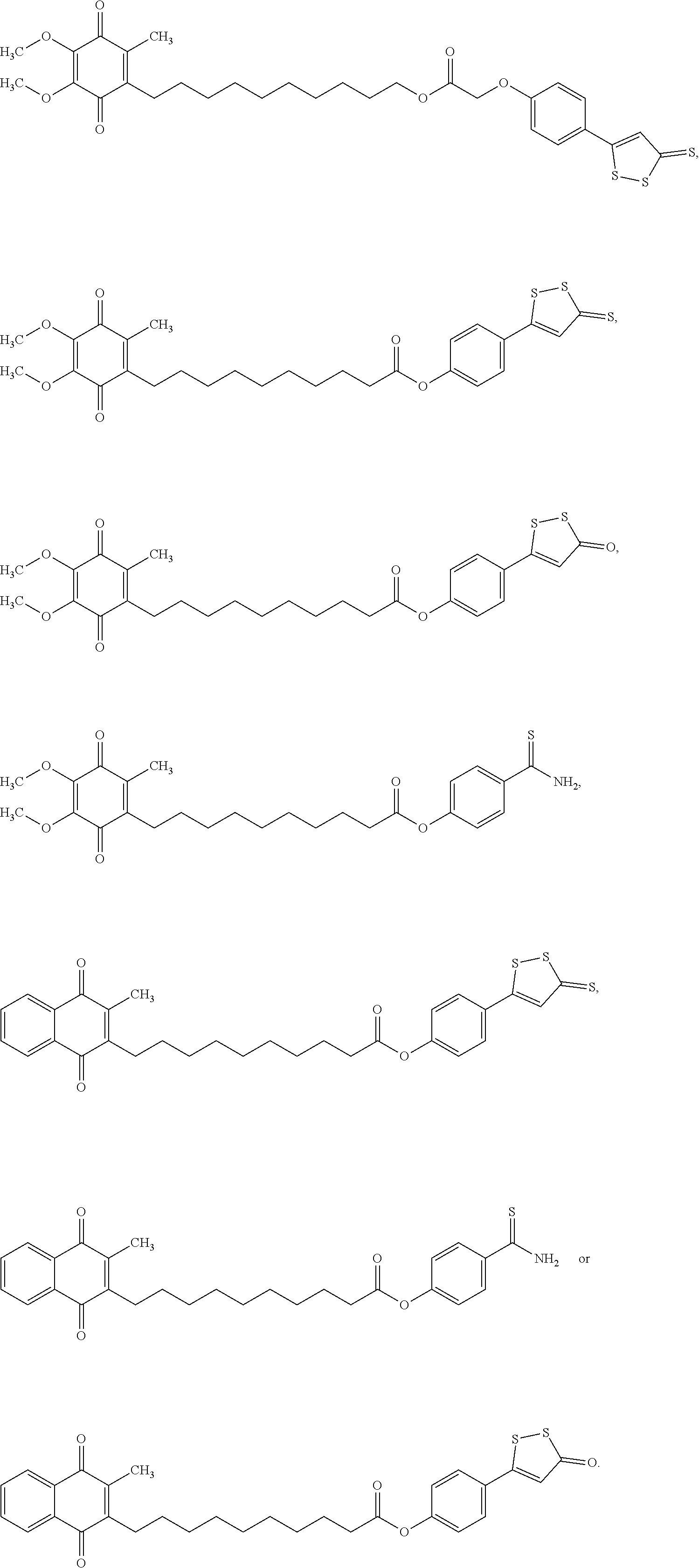

### Chemical Terms

The term “carbo,” “carbyl,” “hydrocarbo,” and “hydrocarbyl,” as used herein, pertain to compounds and/or groups which have only carbon and hydrogen atoms.

The term “hetero,” as used herein, pertains to compounds and/or groups which have at least one heteroatom, for example, multivalent heteroatoms (which are also suitable as ring heteroatoms) such as boron, silicon, nitrogen, phosphorus, oxygen, and sulfur, and monovalent heteroatoms, such as fluorine, chlorine, bromine, and iodine.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “ring,” as used herein, pertains to a closed ring of from 3 to 10 covalently linked atoms, more preferably 3 to 8 covalently linked atoms.

The term “aromatic ring,” as used herein, pertains to a closed ring of from 3 to 10 covalently linked atoms. more preferably 5 to 8 covalently linked atoms, which ring is aromatic.

The term “heterocyclic ring.” as used herein, pertains to a closed ring of from 3 to 10 covalently linked atoms, more preferably 3 to 8 covalently linked atoms, wherein at least one of the ring atoms is a multivalent ring heteroatom, for example, nitrogen, phosphorus, silicon, oxygen, and sulfur, though more commonly nitrogen, oxygen, and sulfur.

The term “alicyclic,” as used herein, pertains to compounds and/or groups which have one ring, or two or more rings (e.g., spiro, fused, bridged), wherein said ring(s) are not aromatic.

The term “aromatic,” as used herein, pertains to compounds and/or groups which have one ring, or two or more rings (e.g., fused), wherein at least one of said ring(s) is aromatic.

The term “heterocyclic,” as used herein, pertains to cyclic compounds and/or groups which have one heterocyclic ring, or two or more heterocyclic rings (e.g., spiro, fused, bridged), wherein said ring(s) may be alicyclic or aromatic.

The term “heteroaromatic,” as used herein, pertains to cyclic compounds and/or groups which have one heterocyclic ring, or two or more heterocyclic rings (e.g., fused), wherein said ring(s) is aromatic. Substituents

The phrase “optionally substituted,” as used herein, pertains to a parent group which may be unsubstituted or which may be substituted.

Unless otherwise specified, the term “substituted,” as used herein, pertains to a parent group which bears one or more substituents. The term “substituent” is used herein in the conventional sense and refers to a chemical moiety which is covalently attached to, appended to, or if appropriate, fused to, a parent group. A wide variety of substituents are well known, and methods for their formation and introduction into a variety of parent groups are also well known.

In one suitable embodiment, the substituent(s), often referred to herein as R, are independently selected from: halo: hydroxy; ether (e.g., C₁₋₇ alkoxy); formyl; acyl (e.g., C₁₋₇alkylacyl, C₅₋₂₀arylacyl); acylhalide; carboxy: ester; acyloxy; amido; acylamido; thioamido; tetrazolyl; amino; nitro; nitroso; azido; cyano; isocyano; cyanato; isocyanato; thiocyano; isothiocyano; sulfhydryl; thioether (e.g., C₁₋₇alkylthio); sulfonic acid; sulfonate; sulfone; sulfonyloxy; sulfinyloxy; sulfamino; sulfonamino; sulfinamino; sulfamyl; sulfonamido; C₁₋₇alkyl (including, e.g., C₁₋₇haloalkyl, C₁₋₇hydroxyalkyl, C₁₋₇carboxyalkyl, C₁₋₇aminoalkyl, C₅₋₂₀aryl-C₁₋₇alkyl); C₃₋₂₀heterocyclyl; or C₅₋₂₀aryl (including, e.g., C₅₋₂₀carboaryl, C₅₋₂₀heteroaryl, C₁₋₇ alkyl-C₅₋₂₀aryl and C₅₋₂chaloaryl)).

In one suitable embodiment, the substituent(s), often referred to herein as R, are independently selected from:
—F, —CI, —Br, and —I;
—OH;
—OMe, —OEt, —O(tBu), and —OCH₂Ph;
—SH;
—SMe, —SEt, —S(tBu), and —SCH₂Ph;
—C(═O)H;
—C(═O)Me, —C(═O)Et, —C(═O)(tBu), and —C(═O)Ph;
—C(═O)OH;
—C(═O)OMe, —C(═O)OEt, and —C(═O)O(tBu);
—C(═O)NH₂—, C(═O)NHMe, —C(═O)NMe₂, and —C(═O)NHEt;
—NHC(═O)Me, —NHC(═O)Et, —NHC(═O)Ph, succinimidyl, and maleimidyl;
—NH₂, —NHMe, —NHEt, —NH(iPr), —NH(nPr), —NMez, —NEt₂, —N(iPr)₂, —N(nPr)₂,
—N(nBu)₂, and —N(tBu)₂;
—CN;
—NO₂;
—Me, —Et, —nPr, —iPr, —nBu, —tBu;
—CF₃—, CHF₂, —CH₂F, —CCl₃—, CBr₃, —CH₂CH₂F, —CH₂CHF₂, and —CH₂CF₃;
—OCF₃—, OCHF₂, —OCH₂F, —OCCI₃—, OCBra, —OCH₂CH₂F, —OCH₂CHF₂, and
—OCH₂CF₃;

—CH₂OH, —CH₂CH₂OH, and —CH(OH)CH₂OH;
—CH₂NH₂, —CH₂CH₂NH₂, and —CH₂CH₂NMez; and, optionally substituted phenyl.

In one suitable embodiment, the substituent(s), often referred to herein as R, are independently selected from: —F, —Cl, —Br, —I, —OH, —OMe, —OEt, —SH, —SMe, -SEt, —C(═O)Me, —C(═O)OH, —C(═O)OMe, —CONHz, —CONHMe, —NH₂, —NMe₂, —NEt₂, —N(nPr)₂, —N(iPr)₂, —CN, —NO₂, —Me, —Et, —CF₃—, OCF₃—, CH₂OH, —CH₂CHzOH, —CH₂NH₂, —CH₂CH₂NH₂, and —Ph.

In one suitable embodiment, the substituent(s), often referred to herein as R, are independently selected from: hydroxy; ether (e.g., C₁₋₇alkoxy); ester; amido; amino; and, C₁₋₇alkyl (including, e.g., C₁₋₇haloalkyl, C₁₋₇zhydroxyalkyl, C₁₋₇carboxyalkyl, C₁₋₇aminoalkyl, C₅₋₂₀aryl-C₁₋₇alkyl).

In one suitable embodiment, the substituent(s), often referred to herein as R, are independently selected from:
—OH;
—OMe, —OEt, —O(tBu), and —OCH₂Ph;
—C(═O)OMe, —C(═O)OEt, and —C(═O)O(tBu);
—C(═O)NH₂, —C(═O)NHMe.—C(═O)NMez, and —C(═O)NHEt;
—NH₂, —NHMe, —NHEt, —NH(iPr), —NH(nPr), —NMez, —NEt₂, —N(iPr)₂, —N(nPr)₂,
—N(nBu)₂, and —N(tBu)₂;
—Me, —Et, —nPr, —iPr, —nBu, —tBu;
—CF₃—, CHF₂, —CH₂F, —CCI₃—, CBr₃—, CH₂CH₂F, —CH₂CHF₂, and —CH₂CF₃;
—CH₂OH, —CH₂CH₂OH, and —CH(OH)CH₂OH; and,
—CH₂NH₂, —CH₂CH₂NH₂, and —CH₂CH₂NMe₂.

The substituents are described in more detail below.

C₁₋₂₀alkyl: The term “C₁₋₂₀alkyl,” as used herein, pertains to a monovalent moiety obtained by removing a hydrogen atom from a C₁₋₇hydrocarbon compound having from 1 to 20 carbon atoms, which may be aliphatic or alicyclic, or a combination thereof, and which may be saturated, partially unsaturated, or fully unsaturated.

Examples of (unsubstituted) saturated linear C₁₋₂₀alkyl groups include, but are not limited to, methyl, ethyl, n-propyl, n-butyl, n-pentyl (amyl), n-octyl, n-nonyl and n-decyl.

Examples of (unsubstituted) saturated branched C₁₋₇alkyl groups include, but are not limited to, iso-propyl, iso-butyl, sec-butyl, tert-butyl, and neo-pentyl.

Examples of saturated alicyclic (also carbocyclic) C₁₋₇alkyl groups (also referred to as “C₃₋₇cycloalkyl” groups) include, but are not limited to, unsubstituted groups such as cyclopropyl, cyclobutyl, cyclopentyl, cyclohexyl, and norbomane, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of (unsubstituted) unsaturated C₁₋₂₀alkyl groups which have one or more carbon-carbon double bonds (also referred to as “C₂₋₇alkenyl” groups) include, but are not limited to, ethenyl (vinyl, —CH=CH₂), 2-propenyl (allyl, —CH—CH═CH₂), isopropenyl (—C(CH₃)═CH₂), butenyl, pentenyl, and hexenyl.

Examples of (unsubstituted) unsaturated C₁₋₂₀alkyl groups which have one or more carbon-carbon triple bonds (also referred to as “C₂₋₂₀alkynyl” groups) include, but are not limited to, ethynyl (ethinyl) and 2-propynyl (propargyl).

Examples of unsaturated alicyclic (also carbocyclic) C₁₋₇alkyl groups which have one or more carbon-carbon double bonds (also referred to as “C₃₋₂₀cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

Additional examples of substituted C₃₋₂₀-cycloalkyl groups include, but are not limited to, those with one or more other rings fused thereto, for example, those derived from: indene (C₉), indan (2,3-dihydro-1H-indene) (C₉), tetraline (1,2,3,4-tetrahydronaphthalene (C₁₀), adamantane (C₁₀), decalin (decahydronaphthalene) (C₁₂), fluorene (C₁₃), phenalene (C₁₃). For example, 2H-inden-2-yl is a Cscycloalkyl group with a substituent (phenyl) fused thereto.

C3.20heterocyclyl: The term “C₃₋₂₀heterocyclyl,” as used herein, pertains to a monovalent moiety obtained by removing a hydrogen atom from a ring atom of a C₃₋₂₀heterocyclic compound, said compound having one ring, or two or more rings (e.g., spiro, fused, bridged), and having from 3 to 20 ring atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆heterocyclyl,” as used herein, pertains to a heterocyclyl group having 5 or 6 ring atoms. Examples of groups of heterocyclyl groups include C₃₋₂₀heterocyclyl, C₃₋₇heterocyclyl, C₅₋₇heterocyclyl.

Examples of (non-aromatic) monocyclic heterocyclyl groups include, but are not limited to, those derived from:

N-i: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₅), azepine (C₇); O₁: oxirane (C₃), oxetane (C₄), oxolane (tetrahydrofuran) (C₅), oxole (dihydrofuran) (C₅), oxane (tetrahydropyran) (C₅), dihydropyran (C₆), pyran (C₆), oxepin (C₇); S-i: thiirane (C₅), thietane (C₄), thiolane (tetrahydrothiophene) (C₅), thiane (tetrahydrothiopyran) (C₆), thiepane (C₇); O₂: dioxolane (C₅), dioxane (C₆), and dioxepane (C₇); O₃: trioxane (C₆); Nz: imidazolidine (C₅), pyrazolidine (diazolidine) (C₅), imidazoline (C₅), pyrazoline (dihydropyrazole) (C₅), piperazine (C₅); N₁O_{1:}tetrahydrooxazole (C₅), dihydrooxazole (C₅), tetrahydroisoxazole (C₅), dihydroisoxazole (C₅), morpholine (C₆), tetrahydrooxazine (C₅), dihydrooxazine (C₅), oxazine (C₆); N₁S₁: thiazoline (C₅), thiazolidine (C₅), thiomorpholine (Ca); N₂O₁: oxadiazine (C₆); O₁S₁: oxathiole (C₅) and oxathiane (thioxane) (C₆); and, N₁O₁St: oxathiazine (C₅).

Examples of substituted (non-aromatic) monocyclic heterocyclyl groups include saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of heterocyclyl groups which are also heteroaryl groups are described below with aryl groups. C₅₋₂₀aryl: The term “C₅₋₂₀aryl,” as used herein, pertains to a monovalent moiety obtained by removing a hydrogen atom from an aromatic ring atom of a C₅₋₂₀aromatic compound, said compound having one ring. or two or more rings (e.g., fused), and having from 5 to 20 ring atoms, and wherein at least one of said ring(s) is an aromatic ring. Preferably, each ring has from 5 to 7 ring atoms. In this context, the prefixes (e.g., C₃₋₂₀, C₅₋₇, C₅₋₆, etc.) denote the number of ring atoms, or range of number of ring atoms, whether carbon atoms or heteroatoms. For example, the term “C₅₋₈aryl,” as used herein, pertains to an aryl group having 5 or 6 ring atoms. Examples of groups of aryl groups include C₃₋₂₀aryl, C₅₋₇aryl, C₅₋₆aryl.

The ring atoms may be all carbon atoms, as in “carboaryl groups” (e.g., C₅₋₂₀carboaryl). Examples of carboaryl groups include, but are not limited to, those derived from benzene (i.e., phenyl) (C₆), naphthalene (C₁₉), azulene (C₁₀), anthracene (C₁₄), phenanthrene (C₁₄), naphthacene (C₁₈), and pyrene (C: 6).

Examples of aryl groups which comprise fused rings, at least one of which is an aromatic ring, include, but are not limited to, groups derived from indene (C₉), isoindene (C₉), and fluorene (C₁₃).

Alternatively, the ring atoms may include one or more heteroatoms, including but not limited to oxygen, nitrogen, and sulfur, as in “heteroaryl groups.” In this case, the group may conveniently be referred to as a “C₅₋₂₀heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of monocyclic heteroaryl groups include, but are not limited to, those derived from:

N₁: pyrrole (azole) (C₅), pyridine (azine) (C₆); O₁: furan (oxole) (C₅); S₁: thiophene (thiole) (C₅); N₁O₁: oxazole (C₅), isoxazole (C₅), isoxazine (C₅); N₂O₁: oxadiazole (furazan) (C₅); NsO₁: oxatriazole (C₅); N₁S₁: thiazole (C₅), isothiazole (C₅); N₂: imidazole (1,3-diazole) (C₅), pyrazole (_{1,2}-diazole) (C₅), pyridazine (1,2-diazine) (C₅), pyrimidine (1,3-diazine) (C₆) (e.g., cytosine, thymine, uracil), pyrazine (1,4-diazine) (C₅); N₃: triazole (C₅), triazine (C₅); and, N₄: tetrazole (C₅).

Examples of heterocyclic groups (some of which are also heteroaryl groups) which comprise fused rings, include, but are not limited to: Coheterocyclic groups (with 2 fused rings) derived from benzofuran (O₁), isobenzofuran (O₁), indole (N₁), isoindole (N₁), purine (N₄) (e.g., adenine, guanine), benzimidazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S); Cioheterocyclic groups (with ₂ fused rings) derived from benzodioxan (O₂), quinoline (N₁), isoquinoline (N₁), benzoxazine (N₁O₁), benzodiazine (N₂), pyridopyridine (N₂), quinoxaline (N₂), quinazoline (N₂); Cisheterocyclic groups (with 3 fused rings) derived from carbazole (N₁), dibenzofuran (O₁), dibenzothiophene (S₁); and, C₁₄heterocyclic groups (with 3 fused rings) derived from acridine (N₁), xanthene (O₁), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂). Heterocyclic groups (including heteroaryl groups) which have a nitrogen ring atom in the form of an —NH-group may be N-substituted, that is, as —NR—. For example, pyrrole may be N-methyl substituted, to give N-methypyrrole. Examples of N-substitutents include, but are not limited to C₁₋₇walkyl, C₃₋₂₀heterocyclyl, C₅₋₂₀aryl, and acyl groups.

Heterocyclic groups (including heteroaryl groups) which have a nitrogen ring atom in the form of an —N═ group may be substituted in the form of an N-oxide, that is, as —N(→O)═ (also denoted —N⁺(→O—)═). For example, quinoline may be substituted to give quinoline N-oxide; pyridine to give pyridine N-oxide; benzofurazan to give benzofurazan N-oxide (also known as benzofuroxan). Cyclic groups may additionally bear one or more oxo (═O) groups on ring carbon atoms. Monocyclic examples of such groups include, but are not limited to, those derived from:

C₅: cyclopentanone, cyclopentenone, cyclopentadienone; C₆: cyclohexanone, cyclohexenone, cyclohexadienone: O₁: furanone (C₅), pyrone (C₆); N₁: pyrrolidone (pyrrolidinone) (C₅), piperidinone (piperidone) (C₆), piperidinedione (C₆) : N₂: imidazolidone (imidazolidinone) (C₅), pyrazolone (pyrazolinone) (C₅), piperazinone (C₅), piperazinedione (C₆), pyridazinone (C₅), pyrimidinone (C₅) (e.g., cytosine), pyrimidinedione (C₆) (e.g., thymine, uracil), barbituric acid (C₆); N₁S₁: thiazolone (C₅), isothiazolone (C₅); N₁O₁: oxazolinone (C₅).

Polycyclic examples of such groups include, but are not limited to, those derived from:

C₉: indenedione; N₁: oxindole (C₉); O₁: benzopyrone (e.g., coumarin, isocoumarin, chromone) (C₁₀) : N₁O_{1:}benzoxazolinone (C₉), benzoxazolinone (C₁₀); N₂: quinazolinedione (C₁₀); N₄: purinone (C₉) (e.g., guanine).

Still more examples of cyclic groups which bear one or more oxo (═O) groups on ring carbon atoms include, but are not limited to, those derived from:
cyclic anhydrides (—C(═O)—O—C(═O)— in a ring), including but not limited to maleic anhydride (C₅), succinic anhydride (C₅), and glutaric anhydride (C₆); cyclic carbonates (—O—C(═O)—O— in a ring), such as ethylene carbonate (C₅) and 1,2-propylene carbonate (C₅); imides (—C(═O)—NR—C(═O)— in a ring), including but not limited to, succinimide (C₅), maleimide (C₅), phthalimide, and glutarimide (C₆); lactones (cyclic esters, —O—C(═O)— in a ring), including, but not limited to, β-propiolactone, γ-butyrolactone, δ-valerolactone (2-piperidone), and s-caprolactone; lactams (cyclic amides, —NR—C(═O)— in a ring), including, but not limited to, β-propiolactam (C₄), γ-butyrolactam (2-pyrrolidone) (C₅), δ-valerolactam (C₅), and —caprolactam (C₇); cyclic carbamates (—O—C(═O) -NR-in a ring), such as 2-oxazolidone (C₅); cyclic ureas (—NR—C(═O)—NR— in a ring), such as 2-imidazolidone (C₅) and pyrimidine-2,4-dione (e.g., thymine, uracil) (C₅).

The above C₁₋₂₀alkyl, C₃₋₂cheterocyclyl, and C₅₋₂₀aryl groups, whether alone or part of another substituent, may themselves optionally be substituted with one or more groups selected from themselves and the additional substituents listed below.

Hydrogen: —H. Note that if the substituent at a particular position is hydrogen, it may be convenient to refer to the compound as being “unsubstituted” at that position.

Halo: —F, —Cl, —Br, and —I.

Hydroxy: —OH.

Ether: —OR, wherein R is an ether substituent, for example, a C₁₋₇alkyl group (also referred to as a C₁₋₇alkoxy group, discussed below), a C₃₋₂₀heterocyclyl group (also referred to as a C₅₋₂₀hetercyclyloxy group), or a Cp₅₋₂₀aryl group (also referred to as a C₅₋₂₀aryloxy group), preferably a C₁₋₇alkyl group.

C₁₋₇alkoxy: —OR, wherein R is a C₁₋₇alkyl group. Examples of C₁₋₇alkoxy groups include, but are not limited to, —OCH₃(methoxy), —OCH₂CH₃ (ethoxy) and —OC(CH₃)₃ (tert-butoxy).

Oxo (keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate: imides, including but not limited to, succinimide and maleimide; lactones (cyclic esters, —O—C(═O)— in a ring), including, but not limited to, β-propiolactone, γ-butyrolactone, δ-valerolactone, and ϵ-caprolactone; and lactams (cyclic amides, —NH—C(═O)— in a ring), including, but not limited to, β-propiolactam, γ-butyrolactam, δ-valerolactam, and ϵ-caprolactam.

Imino (imine): ═NR, wherein R is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of imino groups include, but are not limited to, =NH, =NMe, =NEt, and =NPh.

Formyl (carbaldehyde, carboxaldehyde): —C(═O)H.

Acyl (keto): —C(═O)R, wherein R is an acyl substituent, for example, a C₁₋₂₀alkyl group (also referred to as C₁₋₂₀alkylacyl or C₁₋₂₀alkanoyl), a C₃-zoheterocyclyl group (also referred to as C₅₋₂₀heterocyclylacyl), or a C₅₋₂₀aryl group (also referred to as C₅₋₂₀arylacyl), preferably a C₁₋₂₀alkyl group. Examples of acyl groups include, but are not limited to, —C(═O)CH₃ (acetyl), —C(═O)CH₂CH₃ (propionyl), —C(═O) C(CH₃)3 (butyryl), and —C(═O)Ph (benzoyl, phenone).

Acylhalide (haloformyl, halocarbonyl): —C(═O) X, wherein X is —F, —Cl, —Br, or —I, preferably —Cl, —Br, or —I. Carboxy (carboxylic acid): —COOH.

Ester (carboxylate, carboxylic acid ester, oxycarbonyl): —C(═O)OR, wherein R is an ester substituent, for example, a C₁₋₇alkyl group, a C₃₋₂cheterocyclyl group, or a C₅₋₂₀aryl group, preferably a C₁₋₇alkyl group. Examples of ester groups include, but are not limited to, —C(═O)OCH₃—, C(═O)OCH₂CH₃—, C(═O)OC(CH₃)₃, and—C(═O)OPh.

Acyloxy (reverse ester): —OC(═O) R, wherein R is an acyloxy substituent, for example, a C₁₋₇alkyl group, a C₃₋₂₀heterocyclyl group, or a C₅-zcaryl group, preferably a C₁₋₇alkyl group. Examples of acyloxy groups include, but are not limited to, —OC(═O)CH₃ (acetoxy), —OC(═O)CH₂CH₃—, OC(═O) C(CH₃)₃. —OC(═O)Ph, and —OC(═O)CH₂Ph.

Amido (carbamoyl, carbamyl, aminocarbonyl, carboxamide) : —C(═O)NR¹R², wherein R¹ and R² are independently amino substituents, as defined for amino groups. Examples of amido groups include, but are not limited to, —C(═O)NH₂—, C(═O)NHCH₃—, C(═O)NH(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino) : —NR¹C(═O) R², wherein R¹ is an amide substituent, for example, a C₁₋₇alkyl group, a C₃₋₂₀heterocyclyl group, or a C₅₋₂₀aryl group, preferably a C₁₋₇alkyl group, and R² is an acyl substituent, for example, a C₁₋₇alkyl group, a C₃₋₂₀heterocyclyl group, or a C₅₋₂₀aryl group, preferably a C₁₋₇alkyl group.

Amino: —NR¹R², wherein R¹ and R² are independently amino substituents, for example, hydrogen, a C₁₋₇alkyl group (also referred to as C₁₋₇alkylamino or di-C₁₋₇alkylamino), a C₃₋₂cheterocyclyl group, or a C₅₋₂₀aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridino, azetidino, piperidino, piperazino, morpholino, and thiomorpholino.

Nitro: —NO₂. Nitroso: —NO. Azido: —Na. Cyano (nitrile, carbonitrile): —CN. Isocyano: —NC. Cyanato: —OCN. Isocyanato: —NCO. Thiocyano (thiocyanato): —SCN. Isothiocyano (isothiocyanato): —NCS. Sulfhydryl (thiol, mercapto): —SH.

Thioether (sulfide): —SR, wherein R is a thioether substituent, for example, a C₁₋₇alkyl group (also referred to as a C₁₋₇alkylthio group), a C₃₋₂₀heterocyclyl group, or a C₅₋₂₀aryl group, preferably a C₁₋₇alkyl group. Examples of C₁₋₇alkylthio groups include, but are not limited to, —SCHs and —SCH₂CH₃.

Sulfonic acid (sulfo): —S(═O)₂OH.

Sulfonate (sulfonic acid ester): —S(═O)₂OR, wherein R is a sulfonate substituent, for example, a C₁₋₇alkyl group, a C₃₋₂₀heterocyclyl group, or a C₅₋₂₀aryl group, preferably a C₁₋₇alkyl group. Examples of sulfonate groups include, but are not limited to, —S(═O)₂OCH₃ and —S(═O)₂OCH₂CH₃.

Sulfone (sulfonyl): —S(═O)₂R, wherein R is a sulfone substituent, for example, a C₁₋₇alkyl group, a C₃₋₂₀heterocyclyl group, or a C₅₋₂₀aryl group, preferably a C₁₋₇alkyl group. Examples of sulfone groups include, but are not limited to, —S(═O)₂CHa (methanesulfonyl, mesyl), —S(═O)₂CF₃—, S(═O)₂CHzCH₃, and 4- methylphenylsulfonyl (tosyl).

Sulfonyloxy: —OS(═O)₂R, wherein R is a sulfonyloxy substituent, for example, a C₁₇alkyl group, a C₃₋₂₀heterocyclyl group, or a C₅₋₂₀aryl group, preferably a C₁₋₇alkyl group. Examples of sulfonyloxy groups include, but are not limited to, —OS(═O)₂CH₃ and —OS(═O)₂CH₂CH₃.

Sulfinyloxy: —OS(═O)R, wherein R is a sulfinyloxy substituent, for example, a C₁₋₇alkyl group, a C₃. acheterocyclyl group, or a C₅₋₂₀aryl group, preferably a C₁₋₇alkyl group. Examples of sulfinyloxy groups include, but are not limited to, —OS(═O)CH₃ and —OS(═O)CH₂CH₃.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group.

Examples of sulfonamino groups include, but are not limited to, —NHS(═O)₂CH₃ and —N(CH₃)S(═O)₂C₆Hs.

Sulfinamino: —NR¹S(═O)R, wherein R¹ is an amino substituent, as defined for amino groups, and R is a sulfinamino substituent, for example, a C₁₇alkyl group, a C₃₋₂cheterocyclyl group, or a C₅₋₂₀aryl group, preferably a C₁₋₇alkyl group.

Examples of sulfinamino groups include, but are not limited to, —NHS(═O)CH₃ and —N(CH₃)S(═O)C₆H₅.

Sulfamyl: —S(═O)NR¹R², wherein R¹ and R² are independently amino substituents, as defined for amino groups. Examples of sulfamyl groups include, but are not limited to, —S(═O)NH₂, —S(═O)NH(CHs), —S(═O)N(CH₃)₂, —S(═O)NH(CH₂CH₃), —S(═O)N(CH₂CH₃)₂, and —S(═O)NHPh.

Sulfonamido: —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

As mentioned above, a C₁₋₂₀alkyl group may be substituted with, for example, hydroxy (also referred to as a C₁₋₂chydroxyalkyl group), C₁₋₂₀alkoxy (also referred to as a C₁₋₇alkoxyalkyl group), amino (also referred to as a C₁₋₂caminoalkyl group), halo (also referred to as a C₁₋₂₀haloalkyl group), carboxy (also referred to as a C₁₋₂₀carboxyalkyl group), and C₅₋₂₀aryl (also referred to as a C₅₋₂₀aryl-C₁₋₇alkyl group).

Similarly, a C₅₋₂₀aryl group may be substituted with, for example, hydroxy (also referred to as a C₅₋₂₀hydroxyaryl group), halo (also referred to as a C₅₋₂₀haloaryl group), amino (also referred to as a C₅₋₂₀aminoaryl group, e.g., as in aniline), C₁₋₇alkyl (also referred to as a C₁₋₇alkyl—C₅₋₂₀aryl group, e.g., as in toluene), and C₁₋₇alkoxy (also referred to as a C₁₋₇alkoxy-C₅₋₂₀aryl group, e.g., as in anisole).

These and other specific examples of such substituted groups are also discussed below.

C₁₋₂₀haloalkyl group: The term “C₁₋₂₀haloalkyl group,” as used herein, pertains to a C₁₋₂₀alkyl group in which at least one hydrogen atom (e.g., 1, 2, 3) has been replaced with a halogen atom (e.g., F, Cl, Br, I). If more than one hydrogen atom has been replaced with a halogen atom, the halogen atoms may independently be the same or different. Every hydrogen atom may be replaced with a halogen atom, in which case the group may conveniently be referred to as a C₁₋₂₀perhaloalkyl group.” Examples of C₁₋₇aloalkyl groups include, but are not limited to, —CF₃, —CHF₂, —CH₂F, —CCl₃, —CBr₃, —CH₂CH₂F, —CH₂CHF₂, and —CH₂CF₃.

C₁₋₂₀hydroxyalkyl: The term “C₁-hydroxyalkyl group,” as used herein, pertains to a C₁₋₂₀alkyl group in which at least one hydrogen atom has been replaced with a hydroxy group. Examples of C₁₋₇hydroxyalkyl groups include, but are not limited to, —CH₂OH, —CH₂CH₂OH, and —CH(OH)CH₂OH.

C₁₋₂₀carboxyalkyl: The term “C₁₋₂₀carboxyalkyl group,” as used herein, pertains to a C₁₋₂₀alkyl group in which at least one hydrogen atom has been replaced with a carboxy group. Examples of C₁₋₂₀carboxyalkyl groups include, but are not limited to, —CH₂COOH and —CH₂CH₂COOH.

C₁₋₂₀aminoalkyl: The term “C₁₋₂₀aminoalkyl group,” as used herein, pertains to a C₁₋₂₀alkyl group in which at least one hydrogen atom has been replaced with an amino group. Examples of C₁₋₂₀aminoalkyl groups include, but are not limited to, —CH₂NH₂, —CH₂CH₂NH₂, and —CH₂CH₂N(CH₃)₂.

C₁₋₂₀alkyl—C₅₋₂₀aryl: The term “C₁₋₂₀alkyl—C₅₋₂₀aryl,” as used herein, describes certain C₅₋₂₀aryl groups which have been substituted with a C₁₋₂₀alkyl group. Examples of such groups include, but are not limited to, tolyl (as in toluene), xylyl (as in xylene), mesityl (as in mesitylene), styryl (as in styrene), and cumenyl (as in cumene).

C₅₋₂₀aryl-C₁₋₂₀alkyl: The term “C₅₋₂₀aryl-C₁₋₂₀alkyl,” as used herein, describes certain C₁₋₂₀alkyl groups which have been substituted with a C₅₋₂₀aryl group.

Examples of such groups include, but are not limited to, benzyl (phenylmethyl), tolylmethyl, phenylethyl, and triphenylmethyl (trityl).

C₅₋₂₀haloaryl: The term “C₅₋₂₀haloaryl,” as used herein, describes certain C₅₋₂₀aryl groups which have been substituted with one or more halo groups. Examples of such groups include, but are not limited to, halophenyl (e.g., fluorophenyl, chlorophenyl, bromophenyl, or iodophenyl, whether ortho-, meta-, or para-substituted), dihalophenyl, trihalophenyl, tetrahalophenyl, and pentahalophenyl.

Bidentate Substituents. Some substituents are bidentate, that is, have two points for covalent attachment. For example, a bidentate group may be covalently bound to two different atoms on two different groups, thereby acting as a linker therebetween. Alternatively, a bidentate group may be covalently bound to two different atoms on the same group, thereby forming, together with the two atoms to which it is attached (and any intervening atoms, if present) a cyclic or ring structure. In this way, the bidentate substituent may give rise to a heterocyclic group/compound and/or an aromatic group/compound. Typically, the ring has from 3 to 8 ring atoms, which ring atoms are carbon or divalent heteroatoms (e.g., boron, silicon, nitrogen, phosphorus, oxygen, and sulfur, typically nitrogen, oxygen, and sulfur), and wherein the bonds between said ring atoms are single or double bonds, as permitted by the valencies of the ring atoms. Typically, the bidentate group is covalently bound to vicinal atoms, that is, adjacent atoms, in the parent group.

C₁₋₂₀alkylene: The term “C₁₋₂₀alkylene,” as used herein, pertains to a bidentate moiety obtained by removing two hydrogen atoms, either both from the same carbon atom, or one from each of two different carbon atoms, of a C₁₋₂₀hydrocarbon compound having from 1 to 20 carbon atoms, which may be aliphatic or alicyclic, or a combination thereof, and which may be saturated, partially unsaturated, or fully unsaturated.

Examples of linear saturated C₁₋₂₀alkylene groups include, but are not limited to, —(CH₂)ₙ— where n is an integer from 1 to 20, for example, —CH₂— (methylene), —CH₂CH₂— (ethylene), —CH₂CH₂CH₂-(propylene), and —CH₂CH₂CH₂CH₂-(butylene).

Examples of branched saturated C₁₋₂₀alkylene groups include, but are not limited to, —CH(CH₃)—, —CH(CH₃)CH₂—, —CH(CH₃)CH₂CH₂—, —CH(CH₃)CH₂CH₂CH₂—, -CHECH(CH₃)CH₂-,—CH₂CH(CH₃)CHECH₂—, —CH(CH₂CH₃)—, —CH(CH₂CH₃)CH₂—, and —CH₂CH(CH₂CH₃)CH₂—.

Examples of linear partially unsaturated C₁₋₂₀alkylene groups include, but are not limited to, —CH═CH— (vinylene), —CH═CH—CH₂—, —CH═CH—CH₂—CH₂—, —CH═CH—CH₂—CH₂—CH₂—, —CH═CH—CH═CH—, —CH═CH— CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₂₀alkylene groups include, but are not limited to, —C(CH₃)═CH—, —C(CH₃)=CH—CH₂—, and —CH═CH—CH(CH₃)—.

Examples of alicyclic saturated C₁₋₂₀alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₂₀alkylene groups include, but are not limited to, cyclopentenylene (e.g., 4-cyclopenten-1,3-ylene), cyclohexenylene (e.g., 2-cyclohexen-1,4-ylene, 3-cyclohexen-1,2-ylene, 2,5-cyclohexadien-1,4-ylene).

C₅₋₂₀arylene: The term “C₅₋₂carylene,” as used herein, pertains to a bidentate moiety obtained by removing two hydrogen atoms, one from each of two different ring atoms of a C₅₋₂₀aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboarylene groups,” in which case the group may conveniently be referred to as a “C₅₋₂₀carboarylene” group.

Alternatively, the ring atoms may include one or more heteroatoms, including but not limited to oxygen, nitrogen, and sulfur, as in “heteroarylene groups.” In this case, the group may conveniently be referred to as a “C₅₋₂₀heteroarylene” group, wherein “C₅₋₂₀” denotes ring atoms, whether carbon atoms or heteroatoms.

Preferably, each ring has from 5 to 7 ring atoms, of which from 0 to 4 are ring heteroatoms.

Examples of C₅₋₂₀arylene groups which do not have ring heteroatoms (i.e., C₅₋₂₀carboarylene groups) include, but are not limited to, those derived from benzene (i.e., phenyl) (C₆), naphthalene (C₁₀), anthracene (C-u), phenanthrene (C₁₄), and pyrene (C₁₆).

Examples of C₅₋₂₀heteroarylene groups include, but are not limited to, Csheteroarylene groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and Ceheteroarylene groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

C₅₋₂₀AryleneC₁₋₂₀alkylene: The term “C₅₋₂₀aryleneC₁₋₂₀alkylene,” as used herein, pertains to a bidentate moiety comprising a Cs-20arylene moiety, -Arylene-, linked to a C₁₋₂₀alkylene moiety, -Alkylene-, that is, -Arylene-Alkylene-.

Examples of C₅₋₂₀aryleneC₁₋₂₀alkylene groups include, but are not limited to, phenylene-methylene, phenylene-ethylene, phenylene-propylene, and phenylene-ethenylene (also known as phenylene- vinylene).

C₅₋₂₀AlkyleneC₁₋₂₀arylene: The term “C₅₋₂₀alkyleneC₁₋₂₀arylene,” as used herein, pertains to a bidentate moiety comprising a C₅₋₂₀alkylene moiety, -Alkylene-, linked to a C₁₋₂₀arylene moiety, -Arylene-, that is, -Alkylene-Arylene-.

Examples of C₅₋₂₀alkylene-C₁₋₂₀arylene groups include, but are not limited to, methylene-phenylene, ethylene-phenylene, propylene-phenylene, and ethenylene-phenylene (also known as vinylene- phenylene).

Included in the above are the well known ionic, salt, solvate (e.g., hydrate), and protected forms of these substituents. For example, a reference to carboxylic acid (—COOH) also includes carboxylate (—COO—). Similarly, a reference to an amino group includes a salt, for example, a hydrochloride salt, of the amino group. A reference to a hydroxyl group also includes conventional protected forms of a hydroxyl group.

Similarly, a reference to an amino group also includes conventional protected forms of an amino group.

### Acronyms

For convenience, many chemical moieties are represented herein using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented herein using well known abbreviations, including but not limited to, methanol (MeOH), ethanol (EtOH), iso-propanol (i-PrOH), methyl ethyl ketone (MEK), acetic acid (AcOH), dichloromethane (methylene chloride, DCM), trifluoroacetic acid (TFA), dimethylformamide (DMF), and tetrahydrofuran (THF).

Isomers, Salts, Solvates, Protected Forms, and Prodrugs. A certain compound may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo-and exo-forms; R-, S-, and meso-forms; D- and L-forms; (+) and (−) forms; keto-, enol-, and enolate-forms; syn- and anti-forms; synclinal-and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl.

However, a reference to a class of structures may well include structurally isomeric forms falling within that class (e.g., C₁₋₂₀alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

Unless otherwise specified, a reference to a particular compound includes all such isomeric forms, including racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein in a known manner.

Unless otherwise specified, a reference to a particular compound also includes ionic, salt, solvate (e.g., hydrate), protected forms, and prodrugs thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci. Vol. 66, pp. 1-19.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na+ and K+, alkaline earth cations such as Ca²⁺ and Mg²⁺, and other cations such as Al⁺3. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, ethylenediamine, ethanolamine, diethanolamine, piperazine. An example of a common quaternary ammonium ion is N(CH₃)₄⁺. If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, anions from the following organic acids: acetic, propionic, succinic, gycolic, stearic, lactic, malic, tartaric, citric, ascorbic, maleic, hydroxymaleic, phenylacetic, glutamic, benzoic, salicylic, sulfanilic, 2-acetyoxybenzoic, fumaric, toluenesulfonic, methanesulfonic, ethanesulfonic, ethane disulfonic, oxalic, isethionic, and valeric.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1991), and Protective Groups in Organic Synthesis (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃): a benzyloxy amide (—NHCO—OCH₂C₆H₅, —NH— Cbz); as a t-butoxy amide (—NHCO—OC(CH₃)₃, —NH-Boc); a 2-biphenyl-2-propoxy amide (—NHCO— OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2 (-phenylsulfonyl) ethyloxy amide (—NH-Psec); or, in suitable cases (e.g., cyclic amines), as a nitroxide radical (>N—O·).

For example, a carboxylic acid group may be protected as an ester or an amide, for example, as: a benzyl ester; a t-butyl ester; a methyl ester; or a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug,” as used herein, pertains to a compound which, when metabolised, yields the desired active compound. Typically, the prodrug is inactive, or less active than the active compound, but may provide advantageous handling, administration, or metabolic properties. For example, some prodrugs are esters of the active compound; during metabolysis, the ester group is cleaved to yield the active drug.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Note that specifically included in the term “isomer” are compounds with one or more isotopic substitutions. For example, H may be in any isotopic form, including ¹H, ²H (D), and ³H(T); C may be in any isotopic form, including ¹²C, ¹³C, and ¹⁴C; O may be in any isotopic form, including ¹⁸O and ¹⁸O; and the like.

Unless otherwise specified, a reference to a particular compound includes all such isomeric forms, including racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein in a known manner.

Unless otherwise specified, a reference to a particular compound also includes ionic, salt, solvate (e.g., hydrate), protected forms, and prodrugs thereof, for example, as discussed below.

It may be convenient or desirable to prepare, purify, and/or handle a corresponding salt of the active compound., for example, a pharmaceutically-acceptable salt. Examples of pharmaceutically acceptable salts are discussed in Berge et al., 1977, “Pharmaceutically Acceptable Salts,” J. Pharm. Sci. Vol. 66, pp. 1-19.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na+ and K+, alkaline earth cations such as Ca₂⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, ethylenediamine, ethanolamine, diethanolamine, piperazine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, anions from the following organic acids: acetic, propionic, succinic, gycolic, stearic, lactic, malic, tartaric, citric, ascorbic, maleic, hydroxymaleic, phenylacetic, glutamic, benzoic, salicylic, sulfanilic, 2-acetyoxybenzoic, fumaric, toluenesulfonic, methanesulfonic, ethanesulfonic, ethane disulfonic, oxalic, isethionic, and valeric.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1991), and Protective Groups in Organic Synthesis (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃): a benzyloxy amide (—NHCO—OCH₂C₆H₅, —NH-Cbz); as a t-butoxy amide (—NHCO—OC(CH₃)3, —NH-Boc); a 2-biphenyl-2-propoxy amide (—NHCO—OC(CH₃)₂C₆H₄C₆H5, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2 (-phenylsulfonyl) ethyloxy amide (—NH-Psec); or, in suitable cases (e.g., cyclic amines), as a nitroxide radical (>N—O⋅).

For example, a carboxylic acid group may be protected as an ester or an amide, for example, as: a benzyl ester: a t-butyl ester; a methyl ester; or a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug,” as used herein, pertains to a compound which, when metabolised, yields the desired active compound. Typically, the prodrug is inactive, or less active than the active compound, but may provide advantageous handling, administration, or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### EXAMPLES

### Synthesis of Example 1

Example 1 was prepared in three steps, with an overall yield of 25% (from ADTOH), using the following reaction scheme.

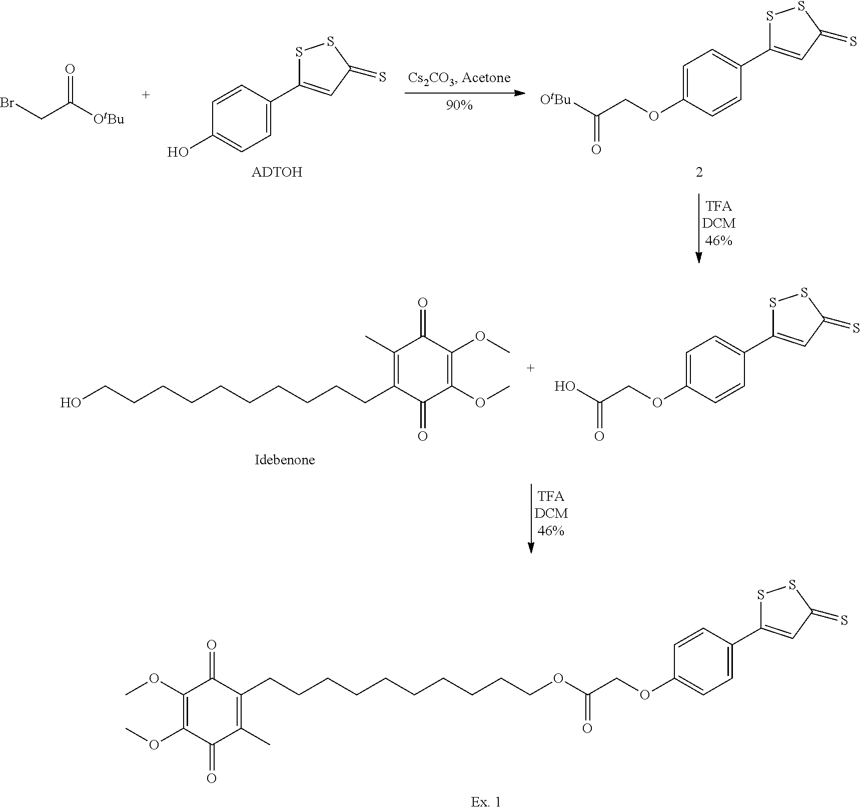

Scheme 1. Example 1 synthetic route. Firstly, 2 (90%) was prepared by a reaction between ADTOH(5-(4-hydroxyphenyl) -3H-1,2-dithiole-3-thione) and tert-butyl bromoacetate (1.5 eq.) with caesium carbonate (2 eq.) in acetone. Following that, the ester protecting group was removed with trifluoroacetic acid (10 eq.) (46% yield). The coupling reaction with idebenone (1 eq.) was carried out using EDCI (1.5 eq.) and DMAP (0.1 eq.) thus, Example 1 was obtained (60% yield).

### Example 2, 3, 4 Syntheses

A more efficient, alternative synthetic approach for bonding ADTOH to idebenone was also carried out. The alcohol functionality in idebenone was oxidised, as previously reported [U.S. Pat. No. 8,263,094], into a carboxylic acid using the Jones reagent (chromic acid made by chromium trioxide or a dichromate salt with sulfuric acid) and a coupling reaction was carried out between ADTOH and the idebenone carboxylic acid to produce Example 2.

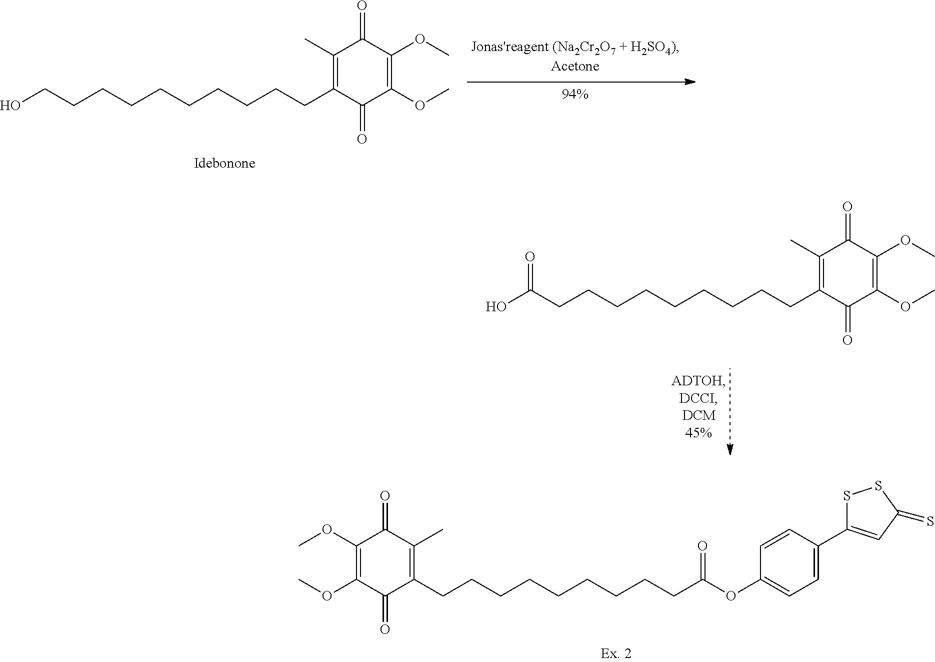 [SMILES: COC1=C(OC)C(=O)C(CCCCCCCCCCO)=C(C)C1=O ; COC1=C(OC)C(=O)C(CCCCCCCCCC(=O)O)=C(C)C1=O ; COC1=C(OC)C(=O)C(CCCCCCCCCC(=O)Oc2ccc(-c3cc(=S)ss3)cc2)=C(C)C1=O]

Scheme 2: Example 2 synthetic route. The idebenone alcohol functionality was oxidised into carboxylic acid (94% yield), using Jones' reagent (16 eq.) made with sodium dichromate dihydrate and sulfuric acid. The product obtained was linked to ADTOH (1 eq.), using EDCl (1.5 eq.) and DMAP (0.1 eq.) to give Example 2 (45% yield).

In an analogous manner, coupling reactions between idebenone carboxylic acid and both HTB (4-hydroxythiobenzamide) and intermediate RT02 were carried out. Example 3 and Example 4 (Scheme 3) were obtained with 45% and 66% overall yields, respectively (from idebenone).

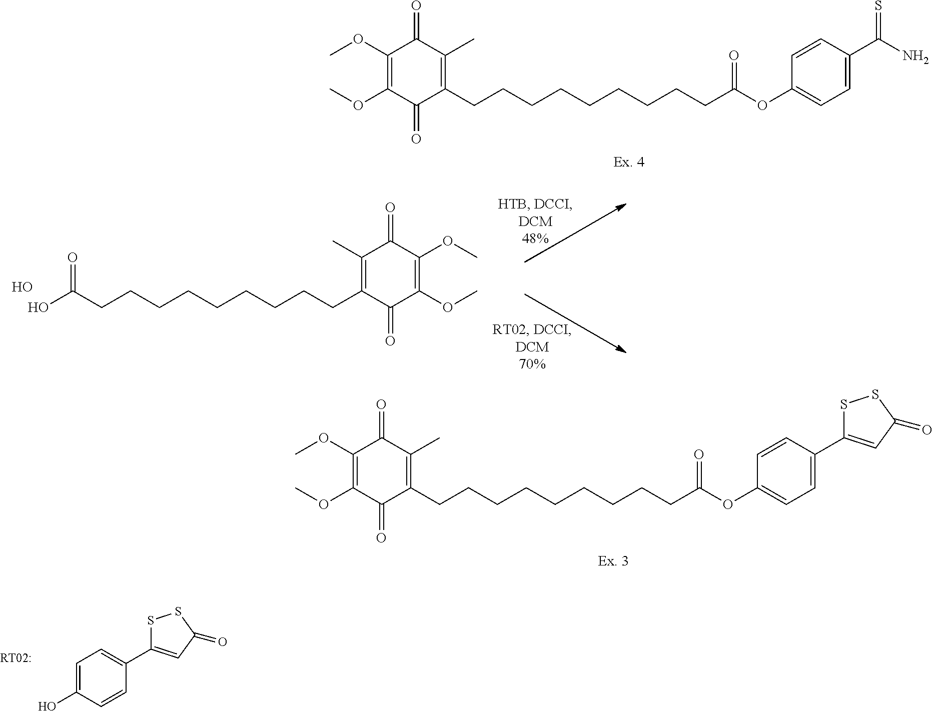

Scheme 3. Example 3 and Example 4 synthetic routes. Idebenone carboxylic acid (1 eq.) was linked to HTB or RT02 (5-(4-hydroxyphenyl) -3H-1,2-dithiol-3-one) using DCCl (N,N′-Dicyclohexylcarbodiimide) (1.5eq.) and DMAP (4-dimethylaminopyridine) (0.1 eq.), producing Example 3 (48% yield) and Example 4 (70% yield).

Example 1 (10-(4,5-dimethoxy-2-methyl-3,6-dioxocyclohexa-1,4-dien-1-yl) decyl (4-3-thioxo-3H-1,2- dithiol-5yl) phenoxy) acetate). In the synthesis of Example 1 silica gel flash chromatography was carried out using a solvent mixture of petroleum ether/ethyl acetate 1/1, which gave RT154 as a red oil (363 mg, 60%, 0.60 mmol) (cLogP=6.23). IR spectrum Vₘₐₓ/cm⁻¹=2851 (m), 1760 (C═O) (m), 1607 (C═O) (s), 1523(s), 1588 (w), 1487 (m), 1436 (m), 1412 (w), 1203 (w), 1184 (m), 1166 (m), 1024 (m), 945 (w), 835 (w), 743(w). ¹H-NMR δ_{H}(400 MHZ, CDCl₃)=7.55 (2H, d, part of AA′BB′, J=8 Hz, aryl CH), 7.31 (1H, s, alkene CH), 6.92 (2H, d, part of AA′BB′, J=12 Hz, aryl CH), 4.63 (2H, s, OCCH₂O), 4.14 (2H, t, J=8 Hz, CH₂O), 3.92 (6H, s, 2×CH₃O), 2.37 (2H, t, J=8 Hz, CH₂), 1.94 (3H, s, CH₃), 1.58 (2H, t, J=8 Hz, CH₂), 1.27-1.19(14H, m, 7×CH₂). ¹³C-NMR δ_{C} (100 MHZ, CDCl₃)=215.20 (C═S), 184.72 (C═O), 184.17 (C═O), 172.57(COO), 168.19 (S—C=CH), 160.99 (aryl CO), 144.30 (C═C), 143.03 (C═C), 138.70 (C═C), 134.94 (alkene CH), 128.64 (aryl CH), 125.15 (aryl CC), 115.59 (aryl CH), 65.80 (OCCH₂), 65.23 (CH₂O), 61.17 (CH₃O), 29.81 (CH₂), 29.46 (CH₂), 29.40 (CH₂), 29.33 (CH₂), 29.14 (CH₂), 28.72 (CH₂), 28.50 (CH₂), 26.39 (CH₂), 25.77 (CH₂), 11.95 (CH₃).

Example 2 ((4-(3-thioxo-3H-1,2-dithiol-5yl) phenoxy)10-(4,5-dimethoxy-2-methyl-3,6-dioxocyclohexa-1,4-dien-1-yl) decanoate). In the synthesis of Example 2 silica gel flash chromatography was carried out using a solvent mixture of petroleum ether/ethyl acetate 2/1, which gave the product as a red oil (252 mg, 45%, 0.45 mmol) (cLogP=6.10). HRMS(ES)⁺ found m/z (rel. intensity)561.1445 (MH⁺; 30), C₂₈H₃₃O₆S₃ requires 561.1439, 226.9663 (MH-idebenone⁺; 100). IR spectrum Vₘₐₓ/cm⁻¹=1762 (C═O) (w), 1705 (C═O) (m), 1642 (s), 1605 (s), 1546 (w), 1436 (m), 1379 (w), 1263 (m), 1204 (m), 1172 (m), 1094 (w), 1025 (w), 836 (w). ¹H-NMR δ_{H}(400 MHZ, CDCl₃)=7.70 (2H, d, part of AA′BB′, J=8 Hz, aryl CH), 7.42 (1H, s, alkene CH), 7.25 (2H, d, part of AA′BB′, J=8 Hz, aryl CH), 4.01 (6H, s, 2×CH₃O), 2.60 (2H, t, J=8 Hz, CH₂), 2.47 (2H, t, J=8 Hz, CH₂), 2.03 (3H, s, CH₃), 1.77 (2H, m, CH₂), 1.42-1.34 (12H, m, 6×CH₂). 13C-NMR δ_{C} (100 MHZ, CDCl₃)=215.51 (C═S), 184.72 (C═O), 184.18 (C═O), 171.78 (COO), 171.72 (S—C═CH), 153.71 (aryl CO), 144.30 (C═C), 143.02 (C═C), 138.71 (C═C), 136.01 (alkene CH), 129.10 (aryl CC), 128.21 (aryl CH), 122.96 (aryl CH), 61.19 (CH₃O), 34.36 (CH₂), 29.79 (CH₂), 29.28 (CH₂), 29.19 (CH₂), 28.72 (CH₂), 26.40 (CH₂), 24.79 (CH₂), 11.95 (CH₃).

Example 3 ((4-carbamothioylphenoxy) l0-(4,5-dimethoxy-2-methyl-3,6-dioxocyclohexa-1,4-dien-1-yl)decanoate). In the synthesis of Example 3, silica gel flash chromatography was carried out using a solvent mixture of ether/ethyl acetate 1/1 and RTI64 was obtained as an orange solid (234 mg, 48%, 0.48 mmol) (cLogP=5.59). HRMS(ES)⁺ found m/z (rel. intensity)488.2095 (MH⁺; 100), C₂₆H₃₄NO6S requires 488.2107. 1H-NMR δ_{H}(400 MHZ, CDCl₃)=7.90 (2H, d, part of AA′BB′, J=8 Hz, aryl CH), 7.84 (1H, br s, NH), 7.55 (1H, br s, NH), 7.10 (2H, d, part of AA′BB′, J=8 Hz, aryl CH), 3.98 (6H, s, 2×CH₃O), 2.57 (2H, t, J=8 Hz, CH₂), 2.44 (2H, t, J=8 Hz, CH₂), 2.01 (3H, s, CH₃), 1.75 (2H, m, CH₂), 1.40-1.32 (12H, m, 6×CH₂). ¹³C-NMR δ_{C} (100 MHZ, CDCl₃)=201.45 (C═S), 184.76 (C═O), 184.23 (C═O), 171.99 (COO), 153.58 (aryl CO), 144.29 (C═C), 144.26 (C═C), 143.05 (C═C), 138.76 (C═C), 136.60 (aryl CC), 128.53 (aryl CH), 121.54 (aryl CH), 61.18 (CH₃O), 34.36 (CH₂), 29.78 (CH₂), 29.24 (CH₂), 29.12 (CH₂), 28.97 (CH₂), 28.72 (CH₂), 26.40 (CH₂), 24.79 (CH₂), 11.95 (CH₃).

Example 4 ((4-(3-oxo-3H-1,2-dithiol-5-yl) phenoxy) l0-(4,5-dimethoxy-2-methyl-3,6-dioxocyclohexa-1,4-dien-1-yl) decanoate). In the synthesis of Example 4, silica gel flash chromatography was carried out using a solvent mixture of petroleum ether/ethyl acetate 2/1. Example 4 was obtained as an orange solid (381 mg, 70%, 0.70 mmol) (cLogP=5.40). HRMS(ES)⁺ found m/z (rel. intensity)545.1663 (MH⁺; 20), C₂₈H₃₃₀₇S2 requires 545.1662, 210.9913 (MH-idebenone+; 100). IR spectrum Vₘₐₓ/cm 1=3295 (m), 1760 (C═O) (w), 1734 (C═O) (w), 1706 (C═O) (w), 1641 (C═O) (s), 1604 (s), 1585 (s), 1457 (m), 1436 (m), 1373 (w), 1263 (m), 1126 (m), 1073 (m), 828 (w), 800 (w), 742 (w). ¹H-NMR δ_{H}(400 MHZ, CDCl₃)=7.58 (2H, d, part of AA′BB′, J=8 Hz, aryl CH), 7.16 (2H, d, part of AA′BB′, J=12 Hz, aryl CH), 6.75 (1H, s, alkene CH), 3.92 (6H, s, 2×CH₃O), 2.52 (2H, t, J=8 Hz, CH₂), 2.38 (2H, t, J=8 Hz, CH₂), 1.94 (3H, s, CH₃), 1.69 (2H, m, CH₂), 1.33-1.25 (12H, m, 6×CH₂). 13C-NMR δ_{C} (100 MHZ, CDCl₃)=194.02 (S—C═O), 184.73 (C═O), 184.19 (C═O), 171.80 (COO), 169.25 (S—C=CH), 153.41 (aryl CO), 144.30 (C═C), 143.03 (C═C), 138.72 (C═C), 130.03 (aryl CC), 127.82 (aryl CH), 122.76 (aryl CH), 118.03 (1H, s, alkene CH), 61.18 (CH₃O), 34.35 (CH₂), 29.80 (CH₂), 29.29 (CH₂), 29.19 (CH₂), 29.04 (CH₂), 28.72 (CH₂), 26.40 (CH₂), 24.80 (CH₂), 11.95 (CH₃).

General procedure for synthesis of Examples 5, 6 and 7. These syntheses were carried out modifying a reported literature protocol [Gerö et al 2016]. 10-(3-methyl-1,4-dioxo-1,4-dihydronaphthalen-2-yl)decanoic acid (292 mg; 0.853 mmol) was dissolved in dichloromethane (8 ml). The mixture was stirred at room temperature and ADTOH(193 mg; 0.853 mmol) or HTB (131 mg; 0.853 mmol) or 5-(4-hydroxyphenyl)-3H-1,2-dithiol-3-one (179 mg; 0.853 mmol) was added to it. 4-Dimethylaminopyridine (10 mg; 0.085 mmol) and N,N-dicyclohexylcarbodiimide (264 mg; 1.28 mmol) (for Examples 5 and 7) or 1-(3-dimethylaminopropyl)-3-ethylcarbodiimide hydrochloride (164 mg; 0.853 mmol) (for Example 6) were added to the initial solution, which was stirred at room temperature for 18 h. The solution was filtered to remove the precipitate formed and the solvent was evaporated in vacuo (for Examples 5 and 7). Alternatively, the reaction mixture was washed with deionised water (6×15 ml), the organic phase was dried over MgSO4 and the solvent was removed under reduced pressure (for Example 6). The crude product obtained was loaded onto a silica gel flash chromatography column.

Synthesis of 10-(3-methyl-1,4-dioxo-1,4-dihydronaphthalen-2-yl) decanoic acid (2-methyl-1,4-naphthoquinone acid derivative). The acid derivative synthesis was carried out by slightly modifying a reported literature protocol [Salmon—Chemin et al 2001]. To a stirred solution containing 2-methyl-1,4-naphthoquinone (300 mg; 1.74 mmol) and undecanedioic acid (1.129 g; 5.22 mmol) in 50 ml of degassed 30% aqueous acetonitrile, silver nitrate (88 mg; 0.522 mmol) was added. A solution of ammonium peroxodisulfate (516 mg; 2.26 mmol) in 12 ml of degassed 30% aqueous acetonitrile was added dropwise to the stirred solution over a 15 minutes period. The resulting solution was stirred under nitrogen atmosphere at 70° C. for 3 h. After cooling the solution to room temperature, the residue was extracted with dichloromethane (3×50 ml) and the organic phases were combined and washed with deionised water (3×50 ml). The organic solution was dried over MgSO4 and the solvent removed under reduced pressure. The crude product was loaded onto a silica gel flash chromatography column, which was eluted with an initial solvent mixture of 3/1 petroleum ether (bp 40-60° C.) /ethyl acetate, followed by 2/1 petroleum ether (bp 40-60° C.) /ethyl acetate solvent mixture to give the title product as yellow solid (292 mg; 49%; 0.853 mmol). 10-(3-methyl-1,4-dioxo-1,4-dihydronaphthalen-2-yl) decanoic acid ¹H-NMR OH(400 MHZ, CDCl₃)=8.01-7.99 (2H, m, aryl CH), 7.63-7.60 (2H, m, aryl CH), 2.55 (2H, t, J=8.0 Hz, CH₂C(O) ), 2.27(2H, t, J=8.0 Hz, CH₂C=C), 2.12 (3H, s, CH₃), 1.58-1.53 (2H, m, CH₂), 1.40-1.23 (12H, m, 6×CH₂). 13C-NMR δ_{C} (100 MHZ, CDCl₃)=185.42 (C═O), 184.75 (C═O), 179.86 (COOH), 147.55 (aryl CC), 143.12 (aryl CC), 133.33 (aryl CH), 133.30 (aryl CH), 132.20 (aryl CC), 126.28 (aryl CH), 126.18 (aryl CH), 34.00 (CH₂), 29.94 (CH₂), 29.33 (CH₂), 29.29 (CH₂), 29.18 (CH₂), 29.01 (CH₂), 28.73 (CH₂), 27.10 (CH₂), 24.64 (CH₂), 12.66 (CH₃).

Example 5 (4-(3-Thioxo-3H-1,2-dithiol-5yl) phenoxy) l0-(1,4-dihydronaphthalen-2-yl) decanoate). In the synthesis of Example 5, the silica gel flash chromatography was carried out using a solvent mixture of petroleum ether (bp 40-60° C.) /ethyl acetate 3/1 and RTK-46 was obtained as an orange waxy solid (179 mg; 38%; 0.324 mmol) (cLogP=7.23). ¹H-NMR δ_{H}(400 MHZ, CDCl₃)=8.02-7.99 (2H, m, aryl CH), 7.63-7.60 (4H, m, aryl CH), 7.33 (1H, s, alkene CH), 7.16 (2H, d, part of AA′BB′, J=8.0 Hz, aryl CH), 2.58-2.50 (4H, m, CH₂C(O) and CH₂C=C), 2.12 (3H, s, CH₃), 1.69-1.67 (2H, m, CH₂), 1.40-1.27 (12H, m, 6×CH₂). ¹³C-NMR δ_{C}(100 MHZ, CDCl₃)=215.52 (C═S), 185.40 (C═O), 184.77 (C═O), 171.76 (COO), 171.71 (aryl CC), 153.71 (aryl CC), 147.51 (aryl CC), 143.14 (aryl CC), 136.00 (alkene CH), 133.36 (aryl CH), 133.34 (aryl CH), 132.20 (aryl CC), 132.17 (aryl CC), 129.09 (aryl CC), 128.20 (aryl CH), 126.28 (aryl CH), 126.21 (aryl CH), 122.95 (aryl CH), 34.36 (CH₂), 29.95 (CH₂), 29.33 (CH₂), 29.30 (CH₂), 29.18 (CH₂), 29.02 (CH₂), 28.74(CH₂), 27.10 (CH₂), 24.79 (CH₂), 12.68 (CH₃).

Example 6 (4-Carbamothioylphenoxy)10-(1,4-dihydronaphthalen-2-yl) decanoate). In the synthesis of Example 6, the silica gel flash chromatography was carried out starting with a solvent mixture of petroleum ether (bp 40-60° C.) /ethyl acetate 2/1 followed by a solvent mixture of petroleum ether (bp 40-60° C.)/ethyl acetate 1/1 and Example 6 was obtained as a yellow solid (98 mg; 24%; 0.205 mmol) (cLogP=6.35). ¹H-NMR δ_{H}(400 MHZ, CDCl₃)=8.00-7.96 (2H, m, aryl CH), 7.81 (2H, d, part of AA′BB′, J=8.0 Hz, aryl CH), 7.75 (1H, br s, NH), 7.61-7.60 (2H, m, aryl CH), 7.41 (1H, br s, NH), 7.02 (2H, d, part of AA′BB′, J=8.0 Hz, aryl CH), 2.56-2.47 (4H, m, CH₂C(O) and CH₂C=C), 2.10 (3H, s, CH₃), 1.69-1.65 (2H, m, CH₂), 1.40-1.26 (12H, m, 6×CH₂). 13C-NMR δ_{C}(100 MHZ, CDCl₃)=201.49 (C═S), 184.76 (C═O), 184.78 (C═O), 171.98 (COO), 171.26 (aryl CC), 153.59 (aryl CC), 147.53 (aryl CC), 143.15 aryl (CC), 136.61 (aryl CC), 133.67 (aryl CH), 133.38 (aryl CC), 128.49 (aryl CH), 126.27 (aryl CH), 126.19 (aryl CH)121.56 (aryl CH), 34.36 (CH₂), 29.93 (CH₂), 29.31 (CH₂), 29.28 (CH₂), 29.15 (CH₂), 29.01 (CH₂), 28.74 (CH₂), 27.09 (CH₂), 24.79 (CH₂), 12.67 (CH₃).

Example 7 (4-(3—Oxo-3H-1,2-dithiol-5-yl) phenoxy) l0-(1,4-dihydronaphthalen-2-yl) decanoate). In the synthesis of Example 7, the silica gel flash chromatography was carried out using a solvent mixture of petroleum ether (bp 40-60° C.) /ethyl acetate 4/1 and RTK-48 was obtained as a yellow solid (128 mg; 28%; 0.239 mmol) (cLogP=6.75). ¹H-NMR δ_{H}(400 MHZ, CDCl₃)=8.00-7.99 (2H, m, aryl CH), 7.63-7.56 (4H, m, aryl CH), 7.15 (2H, d, part of AA′BB′, J=8.0 Hz, aryl CH), 6.74 (1H, s, alkene CH), 2.56-2.50 (4H, m, CH₂C(O) and CH₂C═C), 2.12 (3H, s, CH₃), 1.69-1.57 (2H, m, CH₂), 1.34-1.27 (12H, m, 6×CH₂). ¹³C-NMR δ_{C}(100 MHZ, CDCl₃)=193.99 (S—C═O), 185.39 (C═O), 184.76 (C═O), 171.79 (COO), 169.23 (aryl CC), 153.41 (aryl CC), 147.51 (aryl CC), 143.13 (aryl CC), 133.36 (aryl CH), 133.33 (aryl CH), 132.20 (aryl CC), 132.16 (aryl CC), 130.02 (aryl CC), 127.81 (aryl CH), 126.27 (aryl CH), 126.20 (aryl CH), 122.76 (aryl CH), 118.02 (alkene CH), 34.36 (CH₂), 29.95 (CH₂), 29.33 (CH₂), 29.30 (CH₂), 29.18 (CH₂), 29.02 (CH₂), 28.74 (CH₂), 27.10 (CH₂), 24.80 (CH₂), 12.68 (CH₃).

Synthesis of 2,3-dimethoxy-5-methyl-6-{10-[4-(3-sulfanylidene-3H-1,2-dithiol-5-yl)phenoxy]decyl} cyclohexa-2,5-diene-1,4-dione (Example 8).

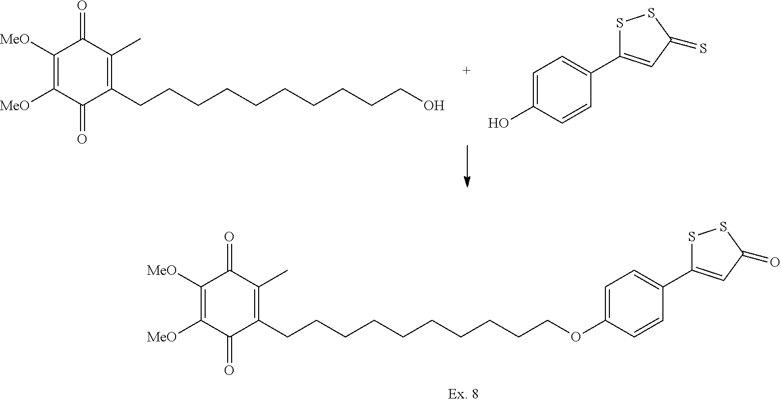 [SMILES: COC1=C(OC)C(=O)C(CCCCCCCCCCO)=C(C)C1=O ; Oc1ccc(-c2cc(=S)ss2)cc1 ; COC1=C(OC)C(=O)C(CCCCCCCCCCOc2ccc(-c3cc(=O)ss3)cc2)=C(C)C1=O]

2-(10-hydroxydecyl) -5,6-dimethoxy-3-methyl-1,4-benzoquinone (1.00 g, 0.00295 mol), and TPP (triphenyl phosphate) (0.775 g, 0.00295 mol) were dissolved in dry THF (12 mL) under nitrogen. DEAD (diethyl azodicarboxylate) (2.2M in toluene) (2.20 mol/L, 1.34 mL, 0.00295 mol) was added dropwise (slightly exotherm noted) and stirred at room temperature for 5 minutes. 5-(4-hydroxyphenyl) dithiole-3-thione (0.669g, 0.00295 mol) was added and stirred at room temperature overnight. The reaction mixture was evaporated to dryness and purified by column chromatography, eluting with DCM. Most impurities were removed. Re-columned in 0-30% EtOAc in hexane to give the product in, Example 8, 18% yield. 1H NMR (400 MHZ, CDCl₃)δ 7.64-7.56 (m, 2H), 7.39 (s, 1H), 7.00-6.92 (m, 2H), 4.02 (t, J=6.5 Hz, 2H), 3.99 (d, J=1.2 Hz, 6H), 2.49-2.39 (m, 2H), 2.01 (d, J=0.7 Hz, 3H), 1.81 (dt, J=14.6, 6.7 Hz, 2H), 1.46 (p, J=6.8 Hz, 2H), 1.42-1.24 (m, 13H). ¹³C NMR (101 MHZ, CDCl₃)δ 215.12 (C), 184.71 (C), 184.17 (C), 173.17 (C), 162.61 (C), 144.34 (C), 144.32 (C), 143.05 (C), 138.69 (C), 134.55 (CH), 128.58 (CH), 123.94 (C), 115.47 (CH), 68.48 (CH₂), 61.15 (CH), 29.81 (CH₂), 29.48 (CH₂), 29.40 (CH₂), 29.33 (CH₂), 29.29 (CH₂), 29.04 (CH₂), 28.72 (CH₂), 26.40 (CH₂), 25.94 (CH₂), 11.91 (CH₃). LCMS 80.5%, @ 225nm (+/−50), MH+547.0

### Synthesis of Example 9

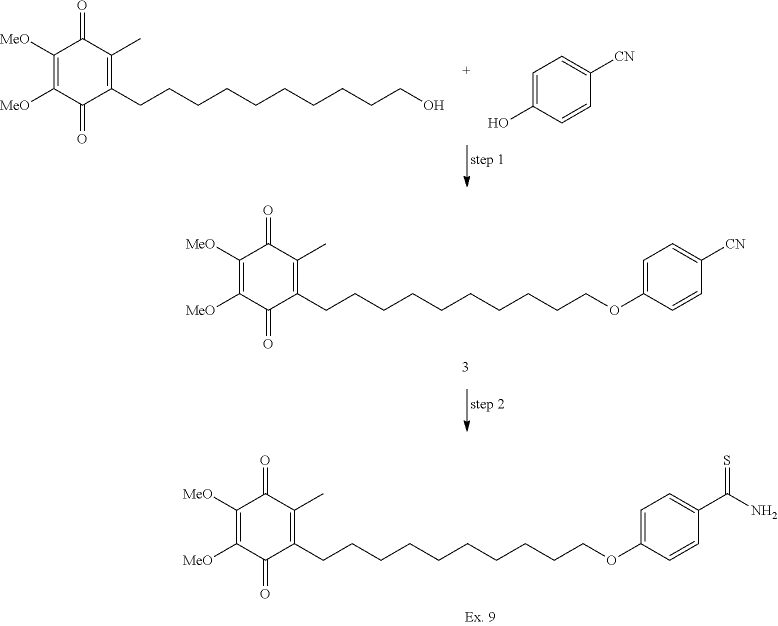 [SMILES: COC1=C(OC)C(=O)C(CCCCCCCCCCO)=C(C)C1=O ; N#Cc1ccc(O)cc1 ; COC1=C(OC)C(=O)C(CCCCCCCCCCOc2ccc(C#N)cc2)=C(C)C1=O ; COC1=C(OC)C(=O)C(CCCCCCCCCCOc2ccc(C(N)=S)cc2)=C(C)C1=O]

Step 1: synthesis of 4-{[10-(4,5-dimethoxy-2-methyl-3,6-dioxocyclohexa-1,4-dien-1-yl)decyl]oxy}benzonitrile, Idebenone (1g, 2.9mol) and 4-hydroxybenzonitrile (352 mg, 2.9 mol, 1 eq) were dissolved in THF (20 ml). After dissolution TPP (852 mg, 3.3 mmol, 1.1 eq) was added, followed by DEAD (2.2 mol sol in toluene, 1.48 ml, 0.00325 mol, 1.1 eq) was added dropwise and the reaction mixture was stirred overnight. The solution was concentrated before purification with ethyl acetate: hexane system to give the product as a red viscous oil which solidified on standing to give the intermediate product 3 as an orange solid, 0.831g, 64% yield. Rf=0.22 (20% ethyl acetate: hexane). ¹H NMR (400 MHZ, CDCl₃)δ 7.61-7.51 (m, 2H), 6.97-6.89 (m, 2H), 3.99 (d, J=1.2 Hz, 9H), 2.49-2.41 (m, 2H), 2.009 (s, 3H), 1.84-1.74 (m, 2H), 1.42-1.23 (m, 14H). LCMS 75%@225nm (+/−50), MH+ 440, 441.

Step 2: Synthesis of 4-{[10-(4,5-dimethoxy-2-methyl-3,6-dioxocyclohexa-1,4-dien-1-yl)decyl]oxy}benzene-1-carbothioamide (compound 9). Magnesium chloride hexahydrate (189 mg, 1.8mmol, 1 eq) and sodium hydrogen sulfide (173 mg, 1.8 mmol, 2 eq) were stirred in THF (5 ml) and to it added intermediate 3 (408 mg, 0.93mmol, 1eq in 5ml THF). This was heated to 30° C., after 1 hour TLC showed complete reaction, and the reaction was concentrated to an oil. The organic phase was then taken up in DCM and eluted through a plug of silica and the relevant spots concentrated. This was then eluted down a Biotage system using 0 to 25% ethyl acetate: hexane to give after concentration the product as an orange solid Example 9, 235 mg, 53% yield. Rf=0.75 (10% ethyl acetate: DCM), 0.13 (20% ethyl acetate: hexane). 1H NMR (400 MHZ, CDCl₃)δ 7.63-7.48 (m, 2H), 7.02-6.85 (m, 2H), 5.28 (d, J=22.5 Hz, 2H), 3.99 (t, J=6.5 Hz, 2H), 3.89 (d, J=1.1 Hz, 6H), 2.64-2.49 (m, 2H), 2.15 (s, 3H), 1.79 (dt, J=14.6, 6.6 Hz, 2H), 1.38 (d, J=53.7 Hz, 14H). ¹³C NMR (CDCl₃, 101 MHZ)δ 162.49 (C), 140.03 (C), 129.83 (C), 136.64 (C), 136.62 (C), 133.96 (CH), 123.21 (C), 119.33 (C), 117.63 (C), 115.20 (CH), 103.64 (C), 68.45 (CH₂), 60.79 (CH₃), 60.73 (CH₃), 29.88 (CH₂), 29.51 (CH₂), 29.48 (CH₂), 29.29 (CH₂), 28.97 (CH₂), 26.34 (CH₂), 25.91 (CH₂), 11.15 (CH₃). LCMS 95% @ 225nm (+/−50), MH^{- 472.}

Example compounds 10-17 were prepared using analogous procedures to those described above for compounds 1-9.

The structures of Example compounds 1-15 are given below.

| Ex- | | |
|---|---|---|
| am- | | |
| ple | Structure | Name |
| 1 | 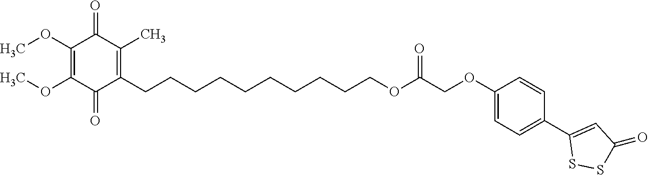 [SMILES: COC1=C(OC)C(=O)C(CCCCCCCCCCOC(=O)COc2ccc(-c3cc(=O)ss3)cc2)=C(C)C1=O] | 10-(4,5-dimethoxy-2- methyl-3,6- dioxocyclohexa-1,4- dien-1-yl)decyl 2-[4-(3-sulfanylidene-3H-1,2- dithiol-5-yl)phenoxy]acetate |
| 2 | 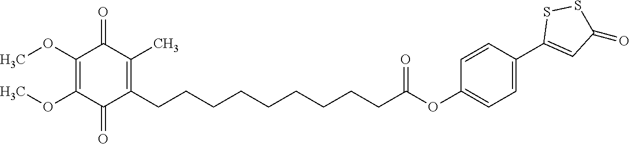 [SMILES: COC1=C(OC)C(=O)C(CCCCCCCCCC(=O)Oc2ccc(-c3cc(=O)ss3)cc2)=C(C)C1=O] | 4-(3-sulfanylidene-3H-1,2- dithiol-5-yl)phenyl) 10-(4,5-dimethoxy-2- methyl-2,6-dioxocyclohexa- 1,4-dien-1-yl)decanoate |
| 3 | 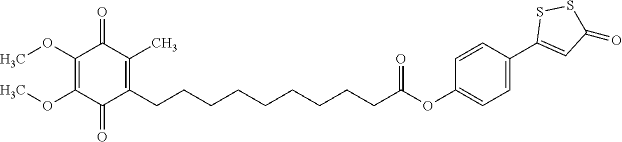 [SMILES: COC1=C(OC)C(=O)C(CCCCCCCCCC(=O)Oc2ccc(-c3cc(=O)ss3)cc2)=C(C)C1=O] | 4-(3-oxo-3H-1,2-dithiol- 5-yl)phenyl 10- (4,5-dimethoxy-2-methyl-3,6- dioxocyclohexa-1,4-dien-1- yl)decanonate |
| 4 | 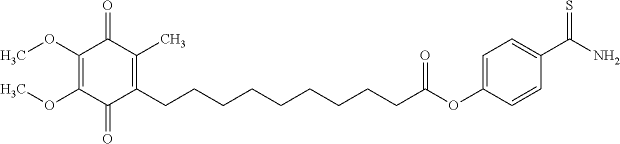 [SMILES: COC1=C(OC)C(=O)C(CCCCCCCCCC(=O)Oc2ccc(C(N)=S)cc2)=C(C)C1=O] | 4-carbamothioylphenyl 10-(4,5- dimethoxy-2-methyl-3,6- dioxocyclohexa-1,4-dien-1- yl)decanoate |
| 5 | 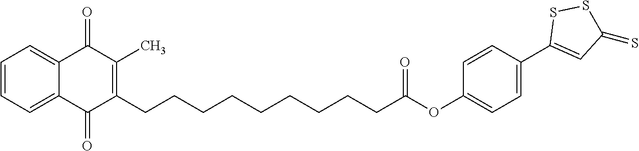 [SMILES: CC1=C(CCCCCCCCCC(=O)Oc2ccc(-c3cc(=S)ss3)cc2)C(=O)c2ccccc2C1=O] | 4-(3-sulfanylidene-3H- 1,2-dithiol-5-yl) phenyl10-(3-methyl-1,4-dioxo- 1,4-dihydronaphthalen-2- yl)decanoate |
| 6 | 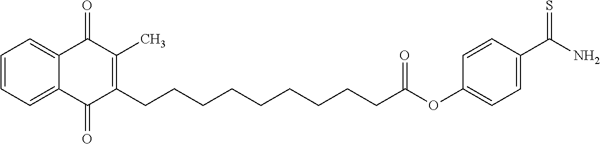 [SMILES: CC1=C(CCCCCCCCCC(=O)Oc2ccc(C(N)=S)cc2)C(=O)c2ccccc2C1=O] | 4-carbamothioylphenyl 10-(3- methyl-1,4-dioxo-1,4- dihydronaphthalen-2-yl) decanoate |
| 7 | 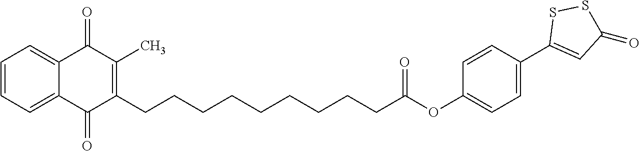 [SMILES: CC1=C(CCCCCCCCCC(=O)Oc2ccc(-c3cc(=O)ss3)cc2)C(=O)c2ccccc2C1=O] | 4-(3-oxo-3H-1,2-dithiol-5- yl)phenyl 10-(3-methyl-1,4-dioxo-1,4- dihydronaphthalen-2-yl) decanoate |
| 8 | 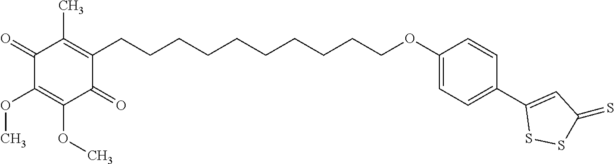 [SMILES: COC1=C(OC)C(=O)C(CCCCCCCCCCOc2ccc(-c3cc(=S)ss3)cc2)=C(C)C1=O] | 2,3-dimethoxy-5-methyl- 6-{10-[4- (3-sulfanylidene-3H- 1,2-dithiol-5-yl) phenoxy]decyl}cyclohexa- 2,5-diene-1,4-dione. |
| 9 | 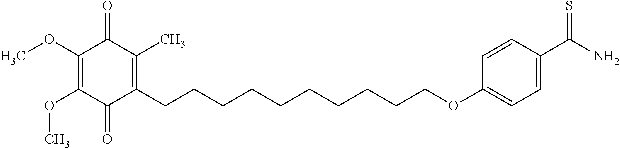 [SMILES: COC1=C(OC)C(=O)C(CCCCCCCCCCOc2ccc(C(N)=S)cc2)=C(C)C1=O] | 4-{[10-(4,5-dimethoxy- 2-methyl- 3,6-dioxocyclohexa-1,4-dien-1- yl)decyl]oxy}benzene-1- carbothioamide |
| 10 | 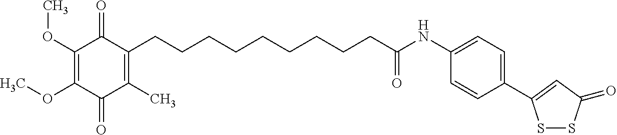 [SMILES: COC1=C(OC)C(=O)C(CCCCCCCCCC(=O)Nc2ccc(-c3cc(=O)ss3)cc2)=C(C)C1=O] | 10-(4,5-dimethoxy-2-methyl- 3,6-dioxocyclohexa-1,4- dien-1-yl)-N-[4-(3-oxo-3H-1,2- dithiol-5- yl)phenyl]decanamide |
| 12 | 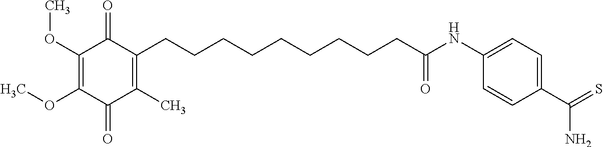 [SMILES: COC1=C(OC)C(=O)C(CCCCCCCCCC(=O)Nc2ccc(C(N)=S)cc2)=C(C)C1=O] | N-(4-carbamothioylphenyl)- 10-(4,5- dimethoxy-2-methyl-3,6- dioxocyclohexa-1,4-dien-1- yl)decanamide |
| 13 | 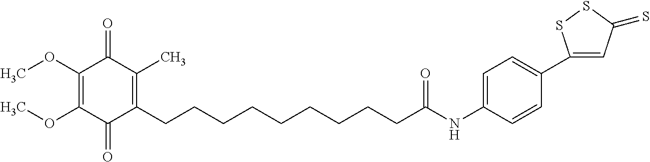 [SMILES: COC1=C(OC)C(=O)C(CCCCCCCCCC(=O)Nc2ccc(-c3cc(=S)ss3)cc2)=C(C)C1=O] | 10-(4,5-dimethoxy-2-methyl- 3,6- dioxocyclohex-1,4-dien-1- yl)-N-[4-(3- sulfanylidene-3H-1,2-dithiol-5- yl)phenyl]decanamide |
| 14 | 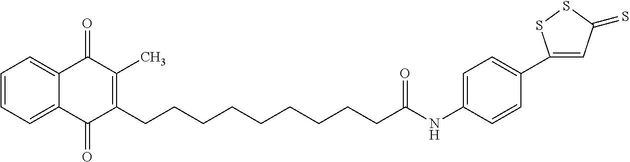 [SMILES: CC1=C(CCCCCCCCCC(=O)Nc2ccc(-c3cc(=S)ss3)cc2)C(=O)c2ccccc2C1=O] | 10-(3-methyl-1,4-dioxo-1,4- dihydronaphthalen-2-yl)-N- [4-(3-sulfanylidene-3H- 1,2-dithiol-5- yl)phenyl]decanamide |
| 15 | 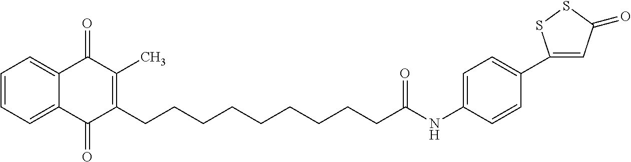 [SMILES: CC1=C(CCCCCCCCCC(=O)Nc2ccc(-c3cc(=O)ss3)cc2)C(=O)c2ccccc2C1=O] | 10-(3-methyl-1,4-dioxo-1,4- dihydronaphthalen-2-yl)-N- [4-(3-oxo-3H-1,2-dithiol-5- yl)phenyl]decanamide |
| 16 | 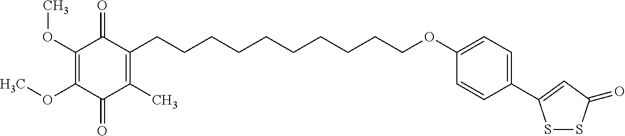 [SMILES: COC1=C(OC)C(=O)C(CCCCCCCCCCOc2ccc(-c3cc(=O)ss3)cc2)=C(C)C1=O] | 2,3-dimethoxy-5- methyl-6-{10-[4- (3-oxo-3H-1,2-dithiol-5-yl) phenoxy]decyl}cyclohexa- 2,5-diene-1,4-dione |
| 17 | 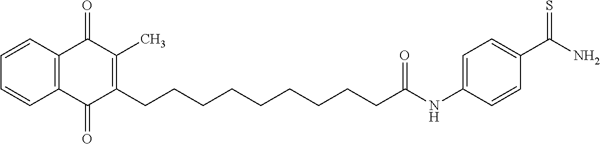 [SMILES: CC1=C(CCCCCCCCCC(=O)Nc2ccc(C(N)=S)cc2)C(=O)c2ccccc2C1=O] | N-(4-carbamothioylphenyl)- 10-(3-methyl-1,4-dioxo-1,4- dihydronaphthalen-2- yl)decanamide |

### Biological Screening Methods And Results

### Cell Culture

b.End3 cells were split by trypsinisation when they reached or slightly exceeded 90% of confluence (usually two times per week). The following procedure was followed: firstly, the old cell medium was removed and the cells were washed with approximately 10 ml of PBS without calcium and magnesium salts. 1 ml of trypsin-EDTA was added to cells, which were rinsed twice with it and then the trypsin solution was removed from the cell flask. Cells were incubated for 2-3 minutes at 37° C., until they detached from the flask surface and finally, fresh supplemented DMEM (4 or 5 ml) was added to the cell flask. The resulting cell-containing mixture was removed from the original flask and it was divided in different flasks with a ratio of 1/4 or 1/5 of the volume. In each flask, more DMEM was added, in order to bring the volume to 12-13 ml. Cell growth and eventual traces of contamination were checked with an inverted microscope every day or every two days.

In order to store the cells for a longer time period and to avoid having to culture them when they were not immediately needed, cells were maintained frozen in liquid nitrogen however, cells were not frozen after passage 27. In order to freeze the cells, the old cell medium was removed and after trypsinisation, they were re-suspended in a solution of cold (4° C.) supplemented DMEM containing 5% of DMSO(Cell Culture Grade) following that, they were transferred (≈10⁻⁶ cells/ml) into cryogenic vials (1.5 ml per each vial) which were placed in a freezing container (Mr. Frosty) filled with isopropyl alcohol to achieve a cooling rate of- 1° C./minute. The freezing container was placed inside a −80° C. freezer, where cells were kept overnight and the following morning, they were transferred to a liquid nitrogen tank. When needed, frozen cells were thawed by warming at 37° C. in a water bath for 1-2 minutes and subsequently, they were gently transferred into a pre-incubated (5 minutes) T75 flask, containing 12 ml of supplemented DMEM. In order to allow the cells to attach to the flask surface, they were incubated overnight and afterwards, the old cell medium was removed and fresh DMEM was added in the flask.

### Hyperglycaemia (HG)-Induced Mitochondrial Dysfunction

b.End3 cell line was used as a model for oxidative stress because in vivo the vascular endothelium was found to be a primary target for oxidative stress. Moreover, in cardiovascular diseases such as hypertension and diabetes a loss of endothelial function and cell death were observed [Poredos et al, 2021]. Also, diabetic microvascular complications (i.e. neuropathy, nephropathy, retinopathy) are mainly caused by an extended exposure of tissue to excessive glucose concentration, which results in an increase of mitochondrial ROS production, changes in mitochondrial membrane potential and loss of ATP synthesis [Vincent et al 2002; Kiritoshi et al, 2003; Manea et al 2004]

In order to induce hyperglycemia in b.End3 cells, a literature reported protocol Lorenzi et al 1985; Qu et al 2014] was followed. After trypsinisation, cells were diluted with supplemented DMEM (approximately 2 ml per flask) and consequently, cells from 3 or 4 different flasks were transferred in a 15 ml sterile conical centrifuge tube and centrifuged for 5 minutes, in order to separate the cells from the medium. Following that, the supernatant medium was removed and the cells were gently re-suspended and mixed with approximately 4 ml of fresh supplemented DMEM. 20 μl of this solution was added to 40 μl of trypan blue and 10 μl of the final solution was loaded onto a cell counting slide dual-chamber. Trypan blue allows to distinguish between live and dead cells. Indeed it is impermeable in live cells, while it is absorbed by dead cells. The number of cells per ml was measured using an automated cell counter. Commonly, the value found was ≈10⁻⁶ cell/ml and consequently, the cell solution was diluted, in order to obtain a concentration of 20,000 cells/well in the minimum volume necessary to fill a 96-well plate (200 μl/well, ≈13 ml for one plate, since the two external columns and rows were loaded with cell-free medium because they are susceptible to evaporation thus, the cell medium in these wells prevented the external cell-containing wells from evaporating). Cells were cultured overnight in a humidified incubator, in order to allow them to attach to the plate surface. Afterwards, the medium was removed (a part of the plate was kept with normal glucose DMEM as a positive control) and supplemented high-glucose DMEM was added. Cells were cultured for 8 days since, in previous studies, it was determined that this is the minimum amount of time required to induce extensive mitochondrial dysfunction, such as excessive oxidant production and mitochondrial membrane hyperpolarisation Lorenzi et al 1985; Qu et al 2014]

On day 8, 10 μl/well (1/20 dilution factor) of a solution of an H₂S donor or of a control compound or a PBS vehicle solution (with calcium and magnesium salts and 10% DMSO) was administered to cells. The final concentration of DMSO in cells was 0.5%: higher DMSO doses may be toxic for the cells. The cells were incubated for a further two days in the presence of the compound and after ten days of hyperglycaemia exposure, the amount of mitochondrial oxidant and mitochondrial membrane hyperpolarisation were determined. Noticeably in this procedure, in order to evaluate the cytoprotective activity of the novel compounds, the drugs were added only after the mitochondrial dysfunction in cells had already occurred. The protocol described is summarised in the scheme below:

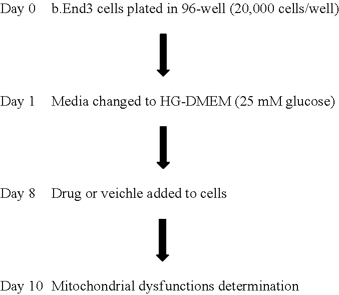

On day 0, cells (20,000 cell/ml) were plated in a 96-well plate. Cells were cultured overnight at 37° C. On day 1, supplemented DMEM was removed and high-glucose supplemented DMEM was added. On day 8, an H₂S donor compound (Examples 1-7), a control compound or altematively a vehicle solution was added to cells. On day 10, mitochondrial dysfunction were determined Lorenzi et al 1985; Qu et al 2014].

### Mitochondrial Superoxide Production

The determination of the mitochondrial superoxide generation was performed with Mitosox Red as previously described in the literature [Mukhopadhyay, 2007]. Mitosox Red is selectively and rapidly taken up by the mitochondria, due to its positive, delocalised charge and its polarity. Inside the organelle, the dye can be easily oxidised by superoxide exhibiting a highly red fluorescence. The dye is selective towards superoxide, and it is not oxidised by nitrogen oxidative species [300].

Oxidation reaction of Mitosox Red by superoxide The oxidised form of Mitosox Red binds to DNA and produces fluorescence [Mukhopadhyay, 2007]:

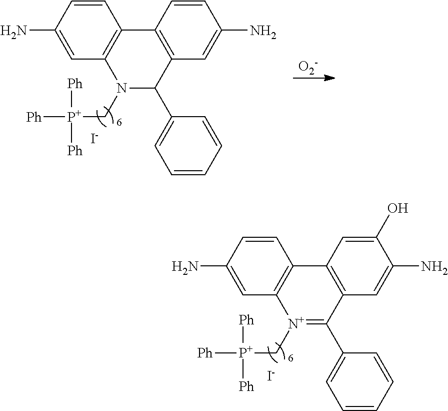

The Mitosox Red protocol was executed as follows: after 10 days of hyperglycemia exposure and two with the test compounds, the medium was removed and endothelial cells were washed twice with 100 μl/well of PBS(with calcium and magnesium salts). They were incubated at 37° C. with 50 μl/well of a 5 μM Mitosox Red solution (5 μl from a 5 mM stock solution in DMSO, added to 5 ml of calcium/magnesium-containing PBS) for 25 minutes. Following that, the cells were washed three times with 100 μl/well of PBS(with calcium/magnesium salts) and loaded with 100 μl/well of reading medium (PBS with calcium/magnesium supplemented, with 10% of FBS). Following that, the oxidation of Mitosox Red (Ex/Em: 510/580 nm) was recorded kinetically on a Pherastar microplate reader at 37° C. using a ROX filter (Ex/Em: 575/610 nm) for 60 minutes. Mitochondrial ROS production is determined as the Vmax value of the fluorescence probe oxidation.

### Results

Table 1 shows the results of this mitochondrial dysfunction screening for the comparative example AP39 and Examples 1-4.

TABLE 1
H₂S donor biologically relevant properties.

| | IC₅₀ | IC₂₅ | | | H-bond |
|---|---|---|---|---|---|
| | (Mitochondrial | (Mitochondrial | | | donor/ |
| Compound | potential | ROS level | TC₅₀ | | H-bond |
| name | restore) (nM) | restore) (nM) | (μM) | cLogP* | acceptor |
| AP39 | 4 | 10 | 8 | 9.48 | 0 |
| | | | | | 2 |
| Example 1 | 0.4 | 6.5 | 147 | 6.23 | 0 |
| | | | | | 7 |
| Example 2 | 6 | 10 | 130 | 6.10 | 0 |
| | | | | | 6 |
| Example 3 | 5 | 10 | 76 | 5.40 | 0 |
| | | | | | 7 |
| Example 4 | 1 | 5 | 100 | 5.59 | 2 |
| | | | | | 7 |

Table 1 shows that the compounds of the invention are as potent, and sometimes more potent, than AP39, and have the advantage of being less toxic. They have a lower clogP which is a predictor of increased aqueous solubility allowing them to be more easily formulated into a medicament. In contrast, AP39 suffers from high hygroscopicity which makes formulation much more difficult than the compounds of the invention.

Worm screening

### Strains and Culture Conditions

C. elegans strains were cultured at 20° C. on Petri dishes containing nematode growth medium (NGM) agar and a lawn of Escherichia coli OP50, unless stated otherwise. Animals for the study were age-synchronized by gravity synchronization from the L1 stage and allowed to grow to the desired day of adulthood. The C. elegans strains used in this study were Bristol strain N2 (WT) provided by the Caenorhabditis Genetics Center.

### Mitochondrial and Cell Death Imaging

Mitochondrial imaging was used in day 1 adults, with or without treatment, to examine the mitochondrial network. Worms were cultured on test compounds as described. Approximately 20 day 1 adults were picked into 20 μL of M9 buffer on a microscope slide with a coverslip applied. Worms were imaged at 40× magnification using a Nikon Eclipse 50i microscope. The protocol used was as described by Oh and Kim. Briefly animals were assessed at day 4 and day 8 of adulthood, where the number of dead muscle cells was determined by quantifying the number of muscle cells that had lost their distinct circular nuclear GFP signal. Approximately 30 animals were picked into 20 μL of M9 buffer on a microscope slide with a coverslip applied. Worms were imaged at 10× magnification by using a Nikon Eclipse 50i microscope.

### Results

FIGS. 1-3 show the results obtained from the worm screening of the test compounds.

FIG. 1 shows the fragmentation of the mitochondrial network in C. elegans caused by ageing: fragmentation is inhibited by Example 2, but not by the constituent parts of the molecule, idebenone and ADTOH.

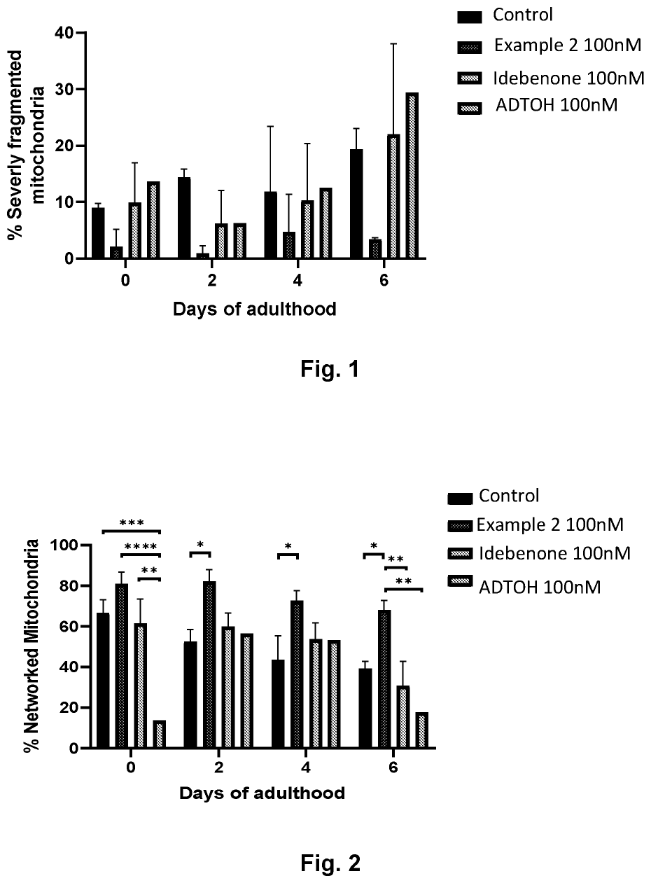

FIG. 2 shows the reduction of networked mitochondria in C. elegans caused by aging. This network is preserved by Example 2 and not by the constituent parts of the molecule idebenone and ADTOH.

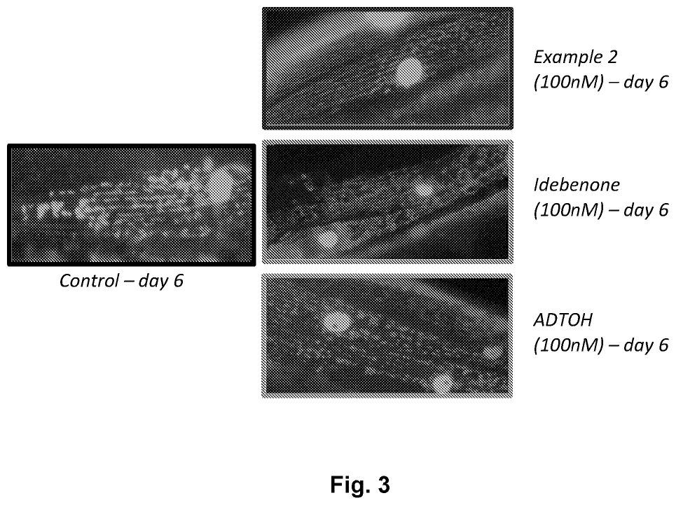

FIG. 3 shows the damage caused to mitochondrial network in aging C. elegans which can be inhibited by Example 2 but not its constituent parts idebenone and ADTOH.

These results clearly show the therapeutic effect of compounds of the invention in the C. elegans model of mitochondrial aging. Thus the compounds may find utility in indications where maintenance of mitochondrial health is therapeutic. In particular C. elegans muscle aging is a well validated animal model for human sarcopenia (reviewed in [Christian and Benian, 2020]).

### Improvement Cellular Bioenergetics in Senescent Primary Human Lung Fibroblasts

Human primary lung fibroblasts were isolated from healthy volunteers and cultured in DMEM supplemented with 10% fetal calf serum and 1% pen/strep and 2 nM L-glutamine, at 37° C., 5% CO₂. To induce senescence, cells were seeded at a density of 50,000 cells per well in 250 μl of cell culture media onto Seahorse V7 plates and treated daily with fresh cell culture media containing 100 nM H₂O2 for 5 days, cells were then cultured with normal medium for a further 9 days. Senescence was confirmed by microscopy and staining for the senescence marker, senescence-associated-β-galactosidase (SA-βgal; Cell Signalling kit #9860). Cells were then treated with compounds (0-300 nM) for a further 24 h (controls are *SIP cells without compounds) and after this time, cellular bioenergetics determined using a Seahorse XFe24 extracellular flux analyser performed as described below. * SIP-senescence-induced phenotype

### Assessment of Cellular Metabolism (Seahorse)—Lung Fibroblasts

For the mitochondrial stress and glycolytic stress tests (Agilent, UK)Seahorse XFe24 sensor cartridges were hydrated with Seahorse XF calibrant solution and maintained at 37° C. in a non-CO₂ incubator overnight. After confirmation of cellular senescence, media was replaced with low buffered Seahorse XF medium supplemented with test example compounds (*0-300 nM) and cells incubated for 1 hr in non-CO₂ incubator, at 37° C. for 1 hr. After incubation, plates were loaded onto Seahorse XFe24 Analyser and basal oxygen consumption rate (OCR) measured for 3 cycles. After basal measurements, cells were injected with the following every 3 cycles: Oligomycin (final concentration 1 μM), FCCP (final concentration 1 μM) and Rotenone/Antimycin A (1:1 ratio, final concentration 0.5 μM). Measurements were taken every 8 minutes on a 3 minute mix, 2 minute wait, 3 minute measure cycle. Extracellular acidification rate (ECAR) and Oxygen consumption rate (OCR) were measured for 3 baseline cycles and injection strategy initiated. Following completion of all assays, media was removed and cells lysed with sodium hydroxide (100 μL per well of 50 mM NaOH). Protein concentrations were quantified using the Bradford method. OCR and ECAR readings were normalised to total protein concentration in each well.

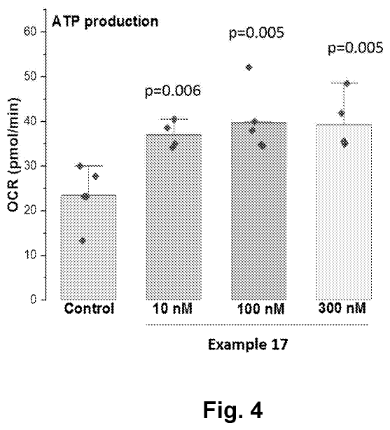

FIG. 4 shows the results for the test compound Example 17. The reduction in the loss of OCR provided by the test compound compared to the control shows that the dysfunction of the mitochondria in these senescent cells is reversed by Example 17. This demonstrates that the compounds of the present invention may provide benefits in ageing cells, in particular ageing airway muscle cells and therefore may provide benefits in the treatment of COPD.

### C2C12 Cell Culture And Differentiation

### Cell Culture

C2C12 myoblast media requirements and seeding densities in Tables 2 and 3, respectively. Cells were maintained sub-confluent (60-70%) in culture in growth medium. For differentiation, cells were plated according to seeding densities and volumes on Table 3, in plating medium, and incubated for 48 hrs at 37° C., 5% CO₂. After 48 hrs (100% confluence) medium was replaced with differentiation medium. Medium was changed every day for 6 consecutive days. Overnight treatments were conducted in serum-deprived amino acid poor medium (Table 2). All experiments were conducted in serum-deprived medium.

TABLE 1
Media composition for C2C12 myoblast culture and differentiation

| Medium | Components |
|---|---|
| Growth medium | High glucose (25 mM) DMEM supplemented with 10% FBS, 4% (v/v) |
| | L-glutamine, 2% penicillin/streptomycin (pen/strep) |
| Plating medium | Glucose-free DMEM with 4% L-glutamine supplemented with 5.5 mM |
| | glucose, 10% FBS and 2% pen/strep |
| Differentiation medium | Glucose-free DMEM with 4% L-glutamine supplemented with 5.5 mM |
| | glucose, 2% horse serum and 2% pen/strep |
| Amino acid-deprived medium | Earl's balanced salt solution 1 x, supplemented with sodium bicarbonate |
| (EBSS) | (2.2 g/L) and 0.34 mM AA solution. |
| Seahorse XF medium - | XF DMEM with 5.5 mM glucose, 2.5 mM sodium pyruvate, 2 mM |
| mitochondrial stress test | L-glutamine, pH 7.4 |

TABLE 2
Seeding density and volumes for C2C12 myoblast culture

| Culture | Density per | Plating | | Differentiation | | Experimental | |
|---|---|---|---|---|---|---|---|
| platform | well/dish | volume | | volume (mL) | | volume (mL) | |
| T75 flask | 1 × 10⁶ | 10 | mL | — | | — | |
| 60 mm dish | 2 × 10⁵ | 5 | mL | 3 | mL | 1.5 | mL |
| F-bottom 96 | 5 × 10³ | 200 | μL | 200 | μL | 50-200 | μL |
| well plate | | | | | | | |
| Seahorse | 3 × 10³ | 200 | μL | 200 | μL | 180 | μL |
| XFe96 | | | | | | | |

microplate

### Assessment of Cellular Metabolism (Seahorse)—Murine C2C12 Skeletal Muscle Myotubes

For the mitochondrial stress tests (Agilent, UK), C2C12 myoblasts were differentiated as described above in Seahorse XFe96 microplates at 3×10³ cells/well. Seahorse XFe96 sensor cartridges were hydrated with Seahorse XF calibrant solution and maintained at 37° C. in a non-CO₂ incubator overnight. On day 7, medium was replaced with low buffered Seahorse XF medium (Table 3) supplemented with test example compounds (*0-300 nM) and cells incubated for 1 hr in non-CO₂ incubator, at 37° C. for 1 hr. After incubation, plates were loaded onto Seahorse XFe96 Analyser and basal oxygen consumption rate (OCR) measured for 4 cycles. After basal measurements, cells were injected with the following every 4 cycles:

Oligomycin (final concentration 2 μM), FCCP (final concentration 1 μM) and Rotenone/Antimycin A (1:1 ratio, final concentration 1 μM). Measurements were taken every 6 minutes on a 3 minute mix, 3 minute measure cycle. Extracellular acidification rate (ECAR) was measured for 4 baseline cycles and injection strategy initiated. Following completion of all assays, media was removed and cells lysed with sodium hydroxide (100 μL per well of 50 mM NaOH). Protein concentrations were quantified using the Bradford method. OCR and ECAR readings were normalised to total protein concentration in each well.

### REFERENCES

Ahmad A and Szabo C(2016) Pharmacol Res 113 (Pt.A):348-355
Ahmad A et al (2016)Shock 45 (1):88-97
Berge S M et al (1977) J Pharm Sci 66 (1):1-19
Chatzianastasiou A et al, (2016) J Pharmacol Exp Ther 358 (3):431-440
Christian, C J and Benian GM (2020) Aging Cell 19 (10):e13223
Covarrubias AE et al (2019) Am J Pathol 189 (1):104-114
Drucker N A et al, (2018) J Pediatr Surg. 53 (9):1692-1698
Ellwod R A et al (2021) Proc Natl Acad Sci USA 118 (9):e2018342118
Escribano-Lopez I et al (2019) Cell Physiol Biochem 52 (2):186-197
Fox B C et al (2020) J Inherit Metab Dis 44 (2):367-375
Gero D et al (2016) Pharmacol Res 113 (pt. A):186-198
Ikeda K et al, (2015)Nitric Oxide 49:90-96
Juriasingani S et al (2018)Nitric Oxide 81:57-66
Karaman Y et al (2021) Basic Clin Pharmacol Toxicol 128 (5):652-660
Karwi Q G et al (2017) Br J Pharmacol 174 (4):287-301
Latorre E et al (2018) Aging 10 (7):1666-1681
Lobb I et al (2017) Am J Transplant 17 (2):341-352
Nishime K et al (2020) Am J Transplant doi: 10.1111/ajt.16401
Oh K. H. and Kim H., Proc. Natl. Acad. Sci. U.S.A. 2013, 110, 19024-19029).
Poredos P et al (2021) Angiology doi: 10.1177/0003319720987752
Salmon-Chemin L et al (2001) J Med Chem 44 (4):548-565
Sanchez-Aranguren L C et al (2020)Sci Rep 10:15810
Sugiyama Y and Fujita T (1985) FEBS Lett. 184 (1):48-51
Szczesny B et al (2014)Nitric Oxide 41:120-130
Tomasova L (2015)Nitric Oxide 46:131-144
Wepler M et al (2019)Shock 52 (2):230-239
Zhao F-L et al (2016)Oxid Med Cell Longev 2016:8360738
Zhu C et al (2019) Am J Transplant 19 (11):3139-3148

1. A compound of formula (I): 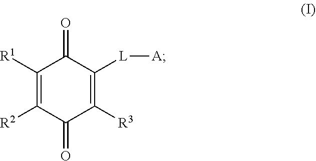
wherein R¹ and R² are independently selected from a C₁₋₆ alkyl group, a C₁₋₆ alkoxy group or together form a cycloalkyl or aryl ring;
wherein R³ is an C₁₋₆ alkyl or C₁₋₆ alkoxy group;
wherein L is a linker group; and
wherein A is a group capable of releasing hydrogen sulphide, or a pharmaceutically acceptable salt thereof.

2. The compound according to claim 1, wherein group A is selected from: 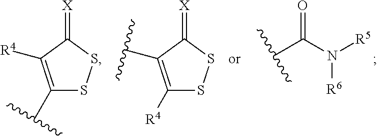
wherein X is S, O or N—OH and R⁴, R⁵ and R⁶ are independently selected from H or C₁₋₇ alkyl groups.

3. The compound according to claim 1, wherein A is selected from a thiocarbamoyl group, a 5-thioxo-5H-1,2-dithiol-3-yl group, a 5-thioxo-5H-1,2-dithiol-4-yl group, a 5-oxo-5H-1,2-dithiol-3-yl group, a 5-oxo-5H-1,2-dithiol-4-yl group, a 5-hydroxyimino-5H-1,2-dithiol-3-yl group, a 5-hydroxyimino-5H-1,2-dithiol-4-yl group, a phosphinodithioate group or a phosphinodithioic acid group.

4. The compound according to claim 1, wherein L comprises a group B which is an optionally substituted alkyl chain, optionally substituted alkenyl chain, or optionally substituted alkynyl chain.

5. The compound according to claim 1, wherein L comprises a group Z selected from a direct bond, —C(═O)NH—, —NHC(═O)—, —O—, —S—, —S(═O)₂NH—, —NHS(═O)₂—, —OC(═O)—, —OC(═O)CH₂O— and —C(═O)O—.

6. The compound according to claim 1, wherein L comprises a group Y which is an optionally substituted 5 or 6 membered cycloalkyl or aryl ring.

7. The compound according to claim 1, having the formula (II): 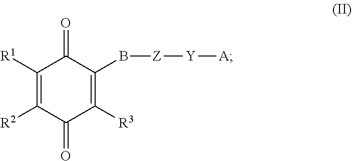
wherein R¹, R² and R³ are as defined in claim 1;
wherein B is an optionally substituted alkyl chain, optionally substituted alkenyl chain, or optionally substituted alkynyl chain;
wherein Z is selected from a direct bond, —C(═O)NH—, —NHC(═O)—, —O—, —S—, −S(═O)₂NH—, —NHS(═O)₂—, —OC(═O)—, —OC(═O)CHO— and —C(═O)O—;
wherein Y is an optionally substituted 5 or 6 membered cycloalkyl or aryl ring; and
wherein A is a group capable of releasing hydrogen sulphide,
or a pharmaceutically acceptable salt thereof.

8. The compound according to claim 1, having the formula (III): 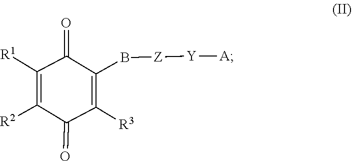
wherein R₁ and R₂ are both C₁₋₆ alkoxy groups or together form a 6-membered aryl ring;
wherein R³ is an C₁₋₆ alkyl group.
wherein B is an optionally substituted a C₆₋₁₄ alkyl chain;
wherein Z is a group selected from —C(═O)NH—, —NHC(═O)—, —O—, —OC(═O)—, —OC(═O)CH₂O—, —OCH₂C(═0)O— and—C(═O)O—;
wherein Y is an optionally substituted phenyl group wherein groups Z and A are attached para to each other on the phenyl group;
wherein A is selected from: 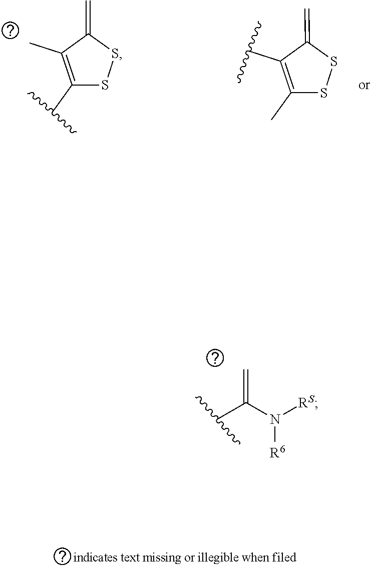
and
wherein X is S, O or N—OH and R⁴, R⁵ and R⁶ are independently selected from H or C₁₋₇alkyl groups;
or a pharmaceutically acceptable salt thereof.

9. The compound according to claim 7 wherein A is selected from a thiocarbamoyl group, a 5-thioxo-5H-1,2-dithiol-3-yl group, a 5-thioxo-5H-1,2-dithiol-4-yl group, a 5-oxo-5H-1,2-dithiol-3-yl group, a 5-oxo-5H-1,2-dithiol-4-yl group, a 5-hydroxyimino-5H-1,2-dithiol-3-yl group, a 5-hydroxyimino-5H-1,2-dithiol-4-yl group, a phosphinodithioate group and a phosphinodithioic acid group.

10. The compound according to claim 1, wherein R¹ and R² are both —OMe and R³ is an C₁₋₃ alkyl group.

11. The compound according to claim 1, wherein R¹ and R² form a 5-or 6-membered aryl ring.

12. The compound according to claim 7, wherein B is an unsubstituted C₁₋₂₀alkyl group.

13. The compound according to claim 7, wherein Z is —C(═O)O— or—OC(═O)CH₂O—.

14. The compound according to claim 7, wherein Y is an optionally substituted phenyl group and wherein groups Z and A are attached para to each other on the phenyl group.

15. The compound according to claim 1 selected from a group consisting of:

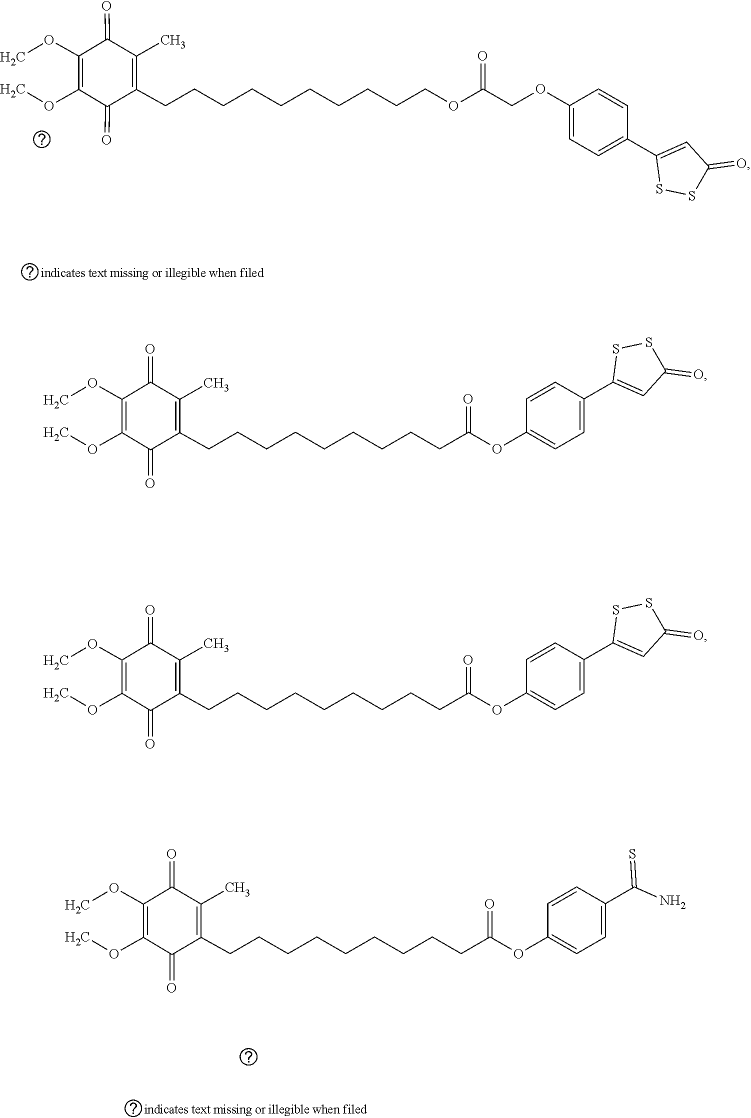 [SMILES: COC1=C(OC)C(=O)C(CCCCCCCCCCOC(=O)COc2ccc(-c3cc(=O)ss3)cc2)=C(C)C1=O ; COC1=C(OC)C(=O)C(CCCCCCCCCC(=O)Oc2ccc(-c3cc(=O)ss3)cc2)=C(C)C1=O ; COC1=C(OC)C(=O)C(CCCCCCCCCC(=O)Oc2ccc(-c3cc(=O)ss3)cc2)=C(C)C1=O ; COC1=C(OC)C(=O)C(CCCCCCCCCC(=O)Oc2ccc(C(N)=S)cc2)=C(C)C1=O]

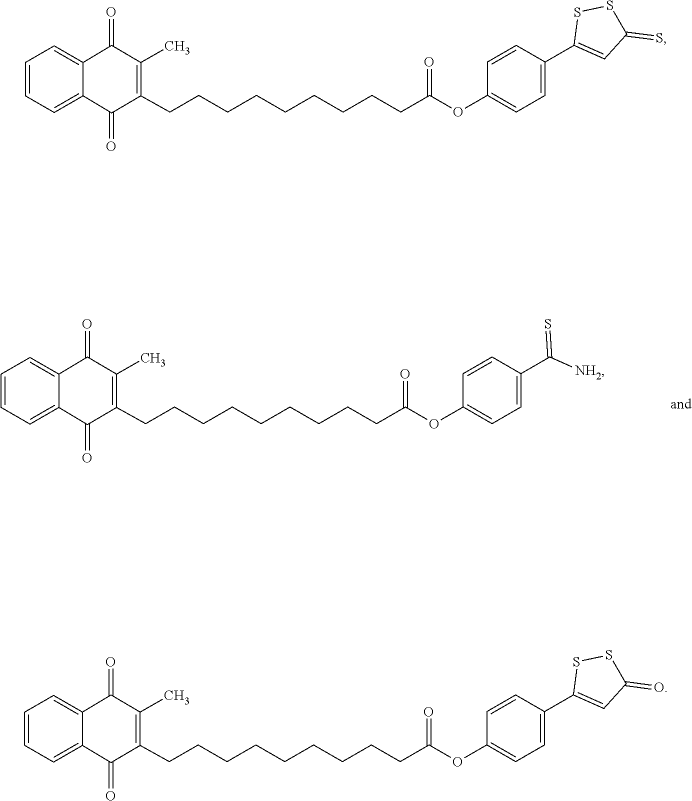 [SMILES: CC1=C(CCCCCCCCCC(=O)Oc2ccc(-c3cc(=S)ss3)cc2)C(=O)c2ccccc2C1=O ; CC1=C(CCCCCCCCCC(=O)Oc2ccc(C(N)=S)cc2)C(=O)c2ccccc2C1=O ; CC1=C(CCCCCCCCCC(=O)Oc2ccc(-c3cc(=O)ss3)cc2)C(=O)c2ccccc2C1=O]

16. The compound according to claim 1 selected from a group consisting of:

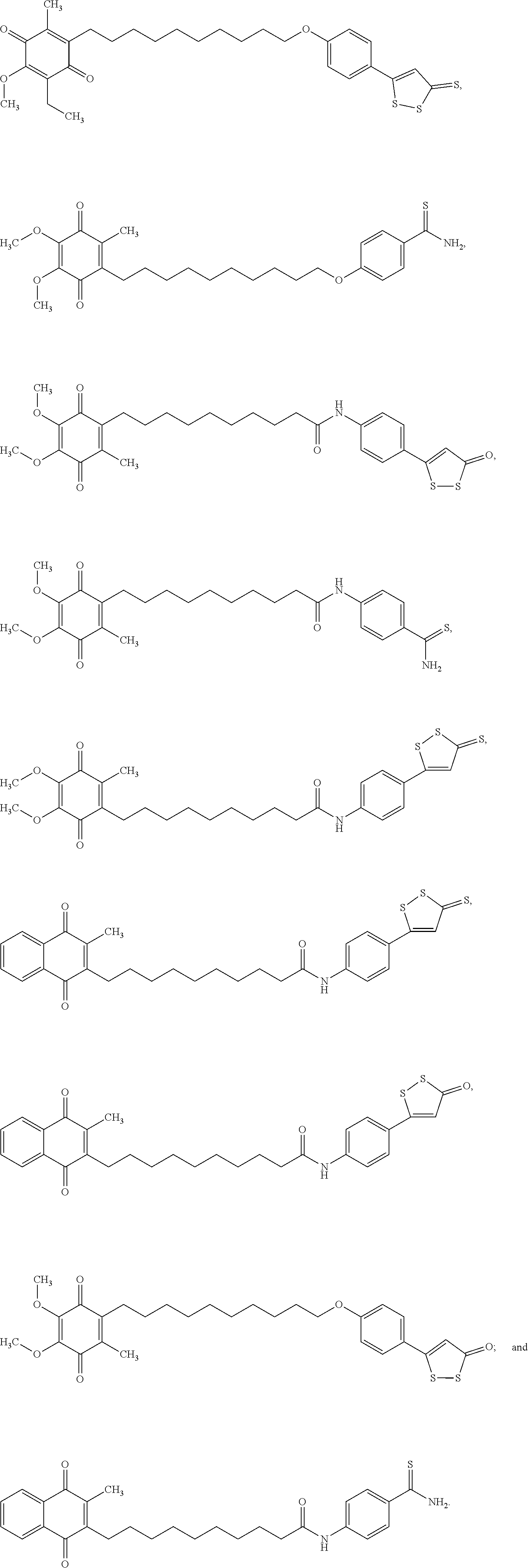

17. (canceled)

18. A method of treating- a neuromuscular or muscular condition in a subject in need thereof, the method comprising administering to the subject a therapeutically effective amount of a compound according to claim 1.

19. The method of claim 18, wherein the neuromuscular or muscular condition is mediated by mitochondrial H₂S donors.

20. The method of claim 18, wherein the neuromuscular or muscular condition is selected from Duchenne Muscular dystrophy, COPD, Leigh syndrome, primary mitochondrial disease, Pancreatic islet transplant, Pre-eclampsia, Cardiac transplant, Renal transplant, Cardiovascular dysfunction, Blunt chest trauma and haemorrhagic shock, Necrotizing enterocolitis, Myocardial reperfusion injury, Burn injury, Diabetic vascular disease, Alzheimer's disease, Acute renal injury, Neurological damage post cardiac arrest and Hypertension.

21. A pharmaceutical composition comprising a compound or a pharmaceutically acceptable salt thereof according to claim 1, and a pharmaceutically acceptable carrier, excipient, or diluent.

22. (canceled)

23. (canceled)
